(19) United States
(12) Patent Application Publication
(10) Pub. No.: US 2025/0169728 A1
(43) Pub. Date: May 29, 2025

(54) SENSOR FOR DETECTING GLUCOSE AND LACTATE AND METHODS FOR DETERMINING AEROBIC AND ANAEROBIC THRESHOLDS

(71) Applicant: ABBOTT DIABETES CARE INC., Alameda, CA (US)

(72) Inventors: Ting Chen, Alameda, CA (US); Junli Ou, Pleasanton, CA (US); James McCarter, St. Louis, MO (US); Matthew Bates, New Hope, PA (US); Paul R. E. Jarvis, Pudsey (GB); Lorelie H. Villarete, Alameda, CA (US)

(73) Assignee: ABBOTT DIABETES CARE INC., Alameda, CA (US)

(21) Appl. No.: 18/821,590

(22) Filed: Aug. 30, 2024

Related U.S. Application Data
(60) Provisional application No. 63/580,252, filed on Sep. 1, 2023.

(51) Int. Cl. A61B 5/1473; A61B 5/00; A61B 5/145

(52) U.S. Cl. CPC A61B 5/14735; A61B 5/14532; A61B 5/14546; A61B 5/4866; A61B 2560/0468

(57) ABSTRACT

The present disclosure describes lactate-responsive sensors, sensing systems incorporating a lactate-responsive sensor, and methods of use thereof that would be beneficial for continuously monitoring lactate levels and determining lactate thresholds (both aerobic and anaerobic thresholds). The present disclosure also relates to an analyte sensor for continuously detecting glucose and lactate levels.

### BACKGROUND

Data-driven training protocols are becoming increasingly important for both world-class athletes and others interested in improving their physical fitness and athletic performance. Lactate concentration in blood or other bodily fluids is often used to determine the fitness level of athletes, prescribe sports trainings, and measure the impact of training in preparation for competition. In particular, anaerobic lactate threshold, which characterizes an individual's aerobic-anaerobic transition zone, is the most widely used metric to guide efficient and effective training especially in endurance sports.

External sensors are available for determining lactate levels, but the results provided by these sensors are often inaccurate. Invasive blood draws can be required to obtain more reliable lactate levels, but this approach can be inconvenient or painful to perform for an individual during a workout routine or training protocol. Moreover, even rapidly performed blood draws (e.g., from the ear lobe) can require at least a brief stoppage in an individual's workout routine, which can lead to a sub-optimal workout efficiency.

Furthermore, glucose monitoring is another important component in sports training which provides athletes with real time information such as energy state, fatigue, and timing of fueling for optimal training.

Thus, there is a need for a sensor that provides continuous lactate monitoring, without the inaccuracies associated with current sensors or the invasive blood draws associated with more accurate technology, and glucose monitoring so that glucose and lactate levels can be tracked in real time to continuously monitor an individual's metabolic profile during sports training and exercise quickly and accurately.

### BRIEF SUMMARY

The present disclosure describes lactate-responsive sensors, sensing systems incorporating a lactate-responsive sensor, and methods of use thereof that are beneficial for monitoring lactate levels and determining lactate thresholds (both aerobic and anaerobic thresholds). These sensors and sensing systems provide a quick and convenient way to obtain reliable lactate levels, including lactate threshold, to provide an individual with valuable information about running pace, heart rate, and/or power that corresponds to an aerobic or anaerobic threshold.

In some embodiments, the present disclosure relates to a sensor that detects both glucose and lactate levels. This sensor can provide continual, real-time feedback on both glucose and lactate levels during training and competition, which can help maximize performance.

The present disclosure relates to an analyte sensor comprising
a substrate;
a first working electrode located on the substrate;
a second working electrode located on the substrate;
a lactate-responsive sensing area disposed on a surface of the first working electrode; and
a glucose-responsive sensing area disposed on a surface of the second working electrode;
a first membrane that is permeable to lactate overcoating the lactate-responsive sensing area; and
a second membrane that is permeable to glucose overcoating the glucose-responsive sensing area and the lactate-responsive sensing area,
wherein the sensor is configured to be partially inserted into an individual's skin.

In some embodiments, the lactate-responsive sensing area can comprise lactate oxidase.

In some embodiments, the lactate-responsive sensing area can comprise a first polymer and a first electron transfer agent.

In some embodiments, the first electron transfer agent can be covalently bonded to the first polymer.

In some embodiments, the glucose-responsive sensing area can comprise glucose oxidase.

In some embodiments, the glucose-responsive sensing area can comprise a second polymer and a second electron transfer agent.

In some embodiments, the second electron transfer agent can be covalently bonded to the second polymer.

In some embodiments, the sensor can further comprise a reference electrode and a counter electrode.

In some embodiments, the first membrane and the second membrane can have different compositions.

The present disclosure also discloses a method for monitoring lactate levels in an individual, comprising exposing an analyte sensor of a sensing system to a fluid, wherein the analyte sensor comprises a substrate; a first working electrode located on the substrate; a second working electrode located on the substrate; a lactate-responsive sensing area disposed on a surface of the first working electrode; and a glucose-responsive sensing area disposed on a surface of the second working electrode; a first membrane that is permeable to lactate overcoating the lactate-responsive sensing area; and a second membrane that is permeable to glucose overcoating the glucose-responsive sensing area and the lactate-responsive sensing area, wherein the sensor is configured to be partially inserted into the individual's skin; applying a potential to the first working electrode of the analyte sensor; obtaining a first signal at or above an oxidation-reduction potential of the lactate-responsive sensing area, the signal being proportional to a concentration of lactate in the fluid; and correlating the signal to the concentration of lactate in the fluid.

In some embodiments, the method can further comprise obtaining a second signal at or above an oxidation-reduction potential of the glucose-responsive sensing area, the signal being proportional to a concentration of glucose in the fluid; and correlating the second signal to the concentration of glucose in the fluid.

In some embodiments, the lactate-responsive sensing area can comprise lactate oxidase.

In some embodiments, the lactate-responsive sensing area can comprise a first polymer and a first electron transfer agent.

In some embodiments, the first electron transfer agent can be covalently bonded to the first polymer.

In some embodiments, the glucose-responsive sensing area can comprise glucose oxidase.

In some embodiments, the glucose-responsive sensing area can comprise a second polymer and a second electron transfer agent.

In some embodiments, the second electron transfer agent can be covalently bonded to the second polymer.

In some embodiments, the sensor can further comprise a reference electrode and a counter electrode.

In some embodiments, the first membrane and the second membrane can have different compositions.

The present disclosure also discloses a method of determining an anaerobic threshold in an individual, comprising continuously measuring signals indicative of lactate concentrations in a biological fluid in the individual with a sensing system comprising a lactate-responsive sensor; communicating the signals indicative of lactate concentrations measured by the lactate-responsive sensor to a processor; and determining an anaerobic threshold based on the signals indicative of lactate concentrations. In some embodiments, the sensor can be any sensor as disclosed herein.

In some embodiments, the individual can be performing a lactate threshold test.

In some embodiments, the lactate threshold test can be a step test with incremental increases in power.

In some embodiments, the anaerobic threshold can be determined by the processor using a broken-stick model.

In some embodiments, the anaerobic threshold can be determined by the processor using a D-max method.

In some embodiments, the anaerobic threshold can be determined by the processor using a modified D-max method.

The present disclosure also discloses a method of determining an aerobic threshold in an individual, comprising continuously measuring signals indicative of lactate concentrations in a biological fluid in the individual with a sensing system comprising a lactate-responsive sensor; communicating the signals indicative of lactate concentrations measured by the lactate-responsive sensor to a processor; determining an anaerobic threshold based on the signals indicative of lactate concentrations. In some embodiments, the sensor can be any sensor as disclosed herein.

In some embodiments, the individual can be performing a lactate threshold test.

In some embodiments, the lactate threshold test can be a step test with incremental increases in power.

In some embodiments, the aerobic threshold can be defined as a fixed value.

In some embodiments, the aerobic threshold can be defined as a baseline lactate concentration plus about 0.5 mM lactate.

In some embodiments, the aerobic threshold can be defined as a baseline lactate concentration plus about 1 mM lactate.

In some embodiments, the aerobic threshold can be determined by the processor using a log-log model.

In some embodiments, the aerobic threshold can be determined by the processor using a segmented regression analysis.

Additional embodiments and advantages of the disclosure will be set forth, in part, in the description that follows, and will flow from the description, or can be learned by practice of the disclosure.

It is to be understood that both the foregoing summary and the following detailed description are exemplary and explanatory only, and do not restrict the scope of the claims.

### BRIEF DESCRIPTION OF THE DRAWINGS/FIGURES

FIG. 1 shows an illustrative plot of lactate levels as a function of variable intensity physical activity.

FIG. 2A shows a diagram of an illustrative sensing system that can incorporate a lactate-responsive sensor of the present disclosure.

FIG. 2B shows a block diagram of a processing electronics that can be associated with one or more components of the sensing system.

FIG. 3A shows a cross-sectional diagram of illustrative two-electrode analyte sensor configuration having a single working electrode.

FIGS. 3B and 3C show diagrams of illustrative three-electrode analyte sensor configurations.

FIG. 3D shows a cross-sectional diagram of illustrative two-electrode analyte sensor configuration having a single working electrode.

FIGS. 3E and 3F show diagrams of illustrative three-electrode analyte sensor configurations.

FIG. 4 shows a cross-section diagram of an analyte sensor having a glucose-responsive sensing area and a lactate-responsive sensing area upon separate working electrodes.

FIG. 5A shows a cross-sectional diagram of an illustrative analyte sensor configuration having two working electrodes, a counter electrode, and a reference electrode.

FIG. 5B shows a cross-sectional diagram of an illustrative analyte sensor configuration having two working electrodes, a counter electrode, and a reference electrode.

FIG. 6 shows an analyte sensor for detecting glucose and lactate according to some embodiments of the present disclosure.

FIG. 7 shows a diagram of an enzyme system that can be used for detecting glucose according to the disclosure herein.

FIG. 8 shows a diagram of an enzyme system that can be used for detecting lactate according to the disclosure herein.

FIG. 9 shows a lactate concentration by power plot for determining an anaerobic threshold by the D-max model.

FIG. 10A shows a lactate concentration by power plot for determining an anaerobic threshold by the broken stick model.

FIG. 10B shows a linear regression model error by power plot for determining an anaerobic threshold by the broken stick model.

FIG. 11 shows a graph of lactate level as detected by the sensor over 128 hours compared to a blood lactate test using a finger prick blood sample or earlobe prick blood sample.

FIG. 12 shows a graph of glucose level as detected by the sensor over 77 hours compared to a blood glucose test using a finger prick blood sample.

FIG. 13 shows a plot of threshold detection by blood lactate test v. threshold detection by a glucose/lactate dual sensor.

FIG. 14 shows a table of the results comparing the anaerobic threshold determinations by a sensor and a blood test.

### DETAILED DESCRIPTION

The headings provided herein are not limitations of the various embodiments of the disclosure, which can be defined by reference to the specification as a whole. It is also to be understood that the terminology used herein is for the purpose of describing particular embodiments only, and is not intended to be limiting, since the scope of the present disclosure will be limited only by the appended claims.

### Definitions

For convenience, the meaning of some terms and phrases used in the specification, examples, and appended claims are provided below. Unless stated otherwise, or implicit from context, the following terms and phrases include the meanings provided below. The definitions are provided to aid in describing particular embodiments, and are not intended to limit the claimed technology, because the scope of the technology is limited only by the claims. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification will control.

The articles “a,” “an,” and “the” are used herein to refer to one or to more than one (i.e., to at least one) of the grammatical object of the article. By way of example, “an element” means one element or more than one element.

As used herein, the term “about” means within an acceptable error range for the particular value as determined by one of ordinary skill in the art, which depends in part on how the value is measured or determined, i.e., the limitations of the measurement system. For example, “about” can mean within 3 or more than 3 standard deviations, per the practice in the art. Alternatively, “about” can mean a range of up to 20% (e.g., up to 10%, up to 5%, or up to 1%) of a given value.

The term “at least” prior to a number or series of numbers is understood to include the number associated with the term “at least,” and all subsequent numbers or integers that could logically be included, as clear from context. When at least is present before a series of numbers or a range, it is understood that “at least” can modify each of the numbers in the series or range. For example, “at least 3” means at least 3, at least 4, at least 5, etc. When at least is present before a component in a method step, then that component is included in the step, whereas additional components are optional.

As used herein, the terms “comprises,” “comprising,” “having,” “including,” “containing,” and the like are open-ended terms meaning “including, but not limited to.” To the extent a given embodiment disclosed herein “comprises” certain elements, it should be understood that present disclosure also specifically contemplates and discloses embodiments that “consist essentially of” those elements and that “consist of” those elements.

As used herein the terms “consists essentially of,” “consisting essentially of,” and the like are to be construed as a semi-closed terms, meaning that no other ingredients which materially affect the basic and novel characteristics of an embodiment are included.

As used herein, the terms “consists of,” “consisting of,” and the like are to be construed as closed terms, such that an embodiment “consisting of” a particular set of elements excludes any element, step, or ingredient not specified in the embodiment.

As used herein, the term “measure” and variations thereof can encompass the meaning of a respective term, such as “determine,” “calculate,” and variations thereof.

As used herein, an “analyte” is an enzyme substrate that is subject to be measured or detected. The analyte can be from, for example, a biofluid and can be tested in vivo, ex vivo, or in vitro.

As used herein, a “sensor” is a device configured to detect the presence and/or measure the level of an analyte in a sample via electrochemical oxidation and reduction reactions on the sensor. These reactions are transduced to an electrical signal that can be correlated to an amount, concentration, or level of an analyte in the sample.

As used herein, a “working electrode” is an electrode at which the analyte (or a second compound whose level depends on the level of the analyte) is electrooxidized or electroreduced with or without the agency of an electron transfer agent.

As used herein, a “counter electrode” refers to an electrode paired with the working electrode, through which passes a current equal in magnitude and opposite in sign to the current passing through the working electrode. In the context of embodiments of the present disclosure, the term “counter electrode” includes both a) counter electrodes and b) counter electrodes that also function as reference electrodes (i.e., counter/reference electrodes), unless otherwise indicated, or unless a reference electrode is also present, in which case the term “counter electrode” is intended to refer solely to a counter electrode.

As used herein, the term “reference electrode” refers to an electrode whose potential is known and can be used as a reference against which the working electrode potential is assessed or measured. In the context of embodiments of the present disclosure, the term “reference electrode” includes both a) reference electrodes and b) reference electrodes that also function as counter electrodes (i.e., counter/reference electrodes), unless otherwise indicated, or unless a counter electrode is also present, in which case the term “reference electrode” is intended to refer only to a reference electrode.

As used herein, components are “immobilized” or “attached” to a polymer and/or a sensor, for example, when the components are entrapped on, entrapped within, covalently bound, ionically bound, electrostatically bound, or coordinatively bound to constituents of a polymer, a sol-gel matric, membrane, and/or sensor, which reduces or precludes mobility.

As used herein an “electron transfer agent” is a compound that carries electrons between the analyte and the working electrode, either directly, or in cooperation with other electron transfer agents. One example of an electron transfer agent is a redox mediator.

As used herein, a “redox mediator” is an electron-transfer agent for carrying electrons between an analyte, an analyte-reduced or analyte-oxidized, enzyme, and an electrode, either directly, or via one or more additional electron-transfer agents. A redox mediator that includes a polymeric backbone can also be referred to as a “redox polymer.”

As used herein, a “sensing area” is a component of the sensor including constituents that facilitate the electrolysis of the analyte. The sensing area can include constituents such as a redox mediator (e.g., an electron transfer agent or a redox polymer), a catalyst (e.g., an analyte-specific enzyme), which catalyzes a reaction of the analyte to produce a response at the working electrode, or both an electron transfer agent and a catalyst. In some embodiments of the present disclosure, a sensor includes a sensing area that is non-leachably disposed in proximity to or on the working electrode. In some embodiments of the present disclosure, the sensing area can be continuously or discontinuously disposed on the working electrode. A sensing area is considered to be “continuously disposed” on the working electrode when the sensing area is applied in a fashion that is uninterrupted across the surface of the working electrode, i.e. a single spot or line, etc. A sensing area is considered to be “discontinuously disposed” on the working electrode when the sensing area is applied as at least two discrete shapes on the working electrode, such as two spots, two lines, a spot and a line, or a plurality (e.g., an array) of spots, lines, or combination thereof. The number of discontinuous applications of the sensing area as a series of spots and/or lines is not considered to be particularly limited, but can range from 2 to about 10 (e.g., 2, 3, 4, 5, 6, 7, 8, 9, or 10, including about 3 to about 8, or from about 4 to about 6). In some embodiments, the sensing area is continuously disposed on the working electrode. In some embodiments, the sensing area is discontinuously disposed on the working electrode.

As used herein, a “sensing element” is an application or region of an analyte-specific enzyme disposed with the sensing area. As such, a sensing element is capable of interacting with the analyte. A sensing area can have more than one sensing element making up the analyte detection area disposed on the working electrode. In some embodiments, the sensing element includes an analyte-specific enzyme and an electron transfer agent (e.g., electron transfer agent). In some embodiments, the sensing element includes an analyte specific enzyme, a redox mediator, and a crosslinker.

As used herein, “crosslinking agent” or “crosslinker” is a molecule that contains at least two (e.g., 2, 3, or 4) reactive groups (e.g., terminal functional groups) that can link at least two molecules together (intermolecular crosslinking) or at least two portions of the same molecule together (intramolecular crosslinking). A crosslinking agent having more than two reactive groups can be capable of both intermolecular and intramolecular crosslinkings at the same time.

The term “patient” refers to a living animal, and thus encompasses a living mammal and a living human, for example. The term “user” can be used herein as a term that encompasses the term “patient.”

Sensors and Sensing systems

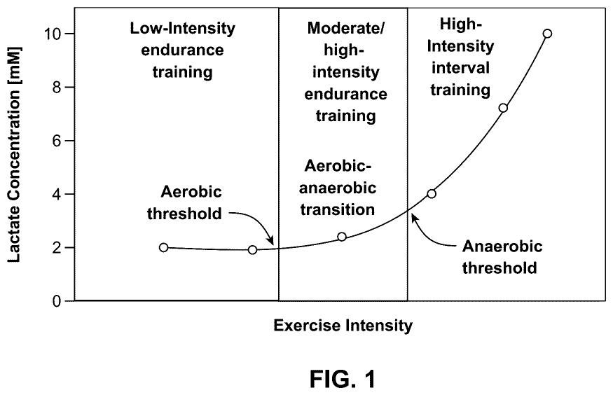

Lactate is produced in vivo during exercise or other activities through glycolytic conversion of glucose, particularly during intense physical activity or exercise. Glycolysis supplies energy to help an individual maintain their current activity level. Lactate levels in an individual are typically characterized as residing within three different zones, as shown in FIG. 1. At lower activity levels (intensities), lactate remains low and the rates of lactate production and lactate clearance remain fairly balanced with one another, such that lactate levels remain relatively constant at or near a fixed baseline concentration, possibly with a slight concentration rise, until a point referred to as the aerobic threshold (LT1) during moderate/hard intensity exercise. After LT1, lactate levels can generally increase linearly until a point referred to as the anaerobic threshold (LT2) during high intensity exercise. After LT2, lactate levels generally show an accelerated increase.

The present disclosure describes lactate-responsive sensors and sensing systems incorporating a lactate-responsive sensor that are beneficial for monitoring lactate levels and determining lactate thresholds, i.e., aerobic threshold and anaerobic threshold. In some embodiments, the lactate-responsive sensor is a sensor that detects both a lactate level and a glucose level. In some embodiments, the system is a continuous lactate monitoring system. In some embodiments, the system is a system for monitoring glucose and lactate. In some embodiments, the system can continuously monitor both glucose and lactate. In some embodiments, a sensor that detects both glucose and lactate levels and a system having the same can provide continual, real-time feedback on both glucose and lactate levels during training and competition, which can help maximize performance.

In some embodiments, the sensor is configured to detect a lactate level. In some embodiments, the sensor is configured to measure a lactate level about every second, about every 3 seconds, about every 5 seconds, about every 10 seconds, about every 15 seconds, about every 20 seconds, about every 30 seconds, about every 45 seconds, about every minute, about every 1.5 minutes, about every 2 minutes, about every 3 minutes, about every 5 minutes, about every 10 minutes, about every 15 minutes, about every 20 minutes, about every 30 minutes, about every 45 minutes, about every hour, about every 2 hours, or about every 3 hours.

In some embodiments, the sensor is configured to communicate a signal that is indicative of the lactate level to a processor. In some embodiments, the sensor can be a sensor as described in the present disclosure. In some embodiments, the sensor can be a sensor as disclosed in U.S. Pat. No. 10,392,647, US 2019/0320947, and/or US 2022/0125354, the disclosures of each of which are incorporated herein by reference in their entirety.

In some embodiments, the system can include various sensing components, such as a processor and/or coding instructions (algorithms) therein, that are adapted to process sensor data received from the lactate sensor and determine a plurality of lactate concentrations therefrom. The processor and/or coding instructions can then analyze the lactate concentrations to determine an aerobic threshold and an anaerobic threshold of the individual.

In some embodiments, the system can comprise a lactate-responsive sensor configured to detect lactate in vivo, and a processor located in a cloud server, remote terminal or a local terminal that is communicatively coupled to the lactate-responsive sensor. Cloud- or server-based communication also falls within the scope of the systems disclosed herein. As used herein, the term “local terminal” refers to a user interface that is physically contiguous with a system where the lactate-responsive sensor is located. For example, in some embodiments, the processor can be contiguous with a housing of the lactate-responsive sensor. As used herein, the term “remote terminal” refers to a user interface that is not located in the same physical space where the lactate-responsive sensor is located. In some embodiments, the remote terminal and its processor can be communicatively coupled to the lactate-responsive sensor or a network. In some embodiments, an individual interfacing with the system can be blinded to the output of the lactate-responsive sensor. In other embodiments, an individual can see the sensor output (e.g., lactate concentrations) in real-time or near real-time, such as on a remote or local viewable display. Remote terminals can include, for example, a dedicated reader device, a dedicated fitness monitoring device (e.g., a FITBIT), a smart phone, or a smart watch.

In some embodiments, the processor can be configured to receive a signal from the lactate-responsive sensor. The processor can be further configured to determine a plurality of lactate concentrations upon receipt of the received signals from the sensor and determine lactate thresholds (both aerobic and anaerobic thresholds) based on the plurality of lactate concentrations. The processor can further signal an individual wearing the sensor or another interested party when predetermined lactate levels have been reached, such as the aerobic threshold and anaerobic threshold, a specified lactate concentration, a multiple of a baseline lactate concentration, or a fraction of a peak lactate concentration, for example. The output of the processor can be numerical and/or graphical. The notification to the wearer of the lactate-responsive sensor or other interested party can be auditory, tactile (haptic), or any combination thereof.

In some embodiments, the active sensing region of the lactate-responsive sensor can be disposed in any suitable location in vivo. Suitable locations can include, but are not limited to, intravenous, subcutaneous, or dermal locations. An intravenous sensor can have the advantage of analyzing lactate directly in blood, but is invasive and can sometimes be painful for an individual to wear over an extended period. Subcutaneous and dermal analyte sensors can often be less painful for an individual to wear due to their shallower penetration and can provide sufficient measurement accuracy in many cases. In some embodiments, the lactate-responsive sensor suitable for use in the present disclosure can be a dermal sensor configured to interrogate dermal fluid of an individual. In some embodiments, the lactate-responsive sensor suitable for use in the present disclosure can be configured to interrogate interstitial fluid of an individual. As used herein, the term “interrogate” refers to the act of measuring a parameter of a sample.

In some embodiments, the sensor can extend from a housing that is configured for external wear upon the skin of an individual performing a given physical activity. The external location where the lactate-responsive sensor is placed is not considered to be particularly limited and can be dependent upon the type of physical activity being performed. In some embodiments, the lactate-responsive sensor can be placed upon the biceps, triceps, upper back, lower back, chest, buttocks, abdomen, thigh, or calf. In some embodiments, multiple lactate-responsive sensors can be used to monitor a single exercise event, such as to perform as a comparison between lactate concentrations measured at two different external locations. One sensor can be located at the site of active muscle usage (e.g., on the thigh during cycling), and the other sensor can be positioned at a location having minimal active muscle usage during the exercise event (e.g., on the arm during cycling), thereby allowing the rate of lactate diffusion from the blood stream into other interstitial tissues to be determined. If desired, the outputs from one or both sensor locations can also be cross-referenced with blood lactate readings obtained from finger or ear lobe pricks.

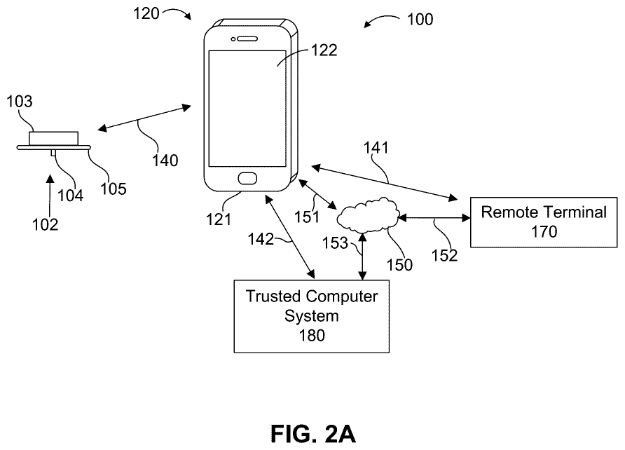

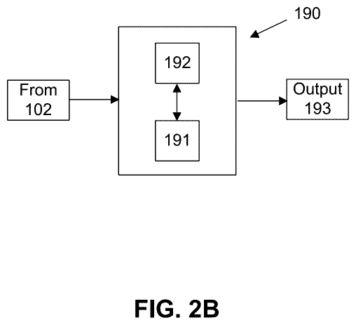

FIG. 2A shows a diagram of an illustrative system that can incorporate a lactate-responsive sensor of the present disclosure. As shown, system 100 includes sensor control device 102 and reader device 120 that are configured to communicate with one another over a local communication path or link 140, which can be wired or wireless, uni- or bi-directional, and encrypted or non-encrypted. Reader device 120 can constitute an output medium for viewing lactate concentrations and alerts or notifications determined by sensor 104 or a processor associated therewith, as well as allowing for one or more user inputs, according to some embodiments. Alternately, reader device 120 can produce output that is blinded to a user. Reader device 120 can be a multi-purpose smartphone or a dedicated electronic reader instrument. While only one reader device 120 is shown, multiple reader devices 120 can be present in certain instances. A suitable processor can also be incorporated in reader device 120, according to some embodiments. Reader device 120 can also be in communication with remote terminal 170 and/or trusted computer system 180 via communication path(s)/link(s) 141 and/or 142, respectively, which also can be wired or wireless, uni- or bi-directional, and encrypted or non-encrypted. Reader device 120 can also or alternately be in communication with network 150 (e.g., a mobile telephone network, the internet, or a cloud server) via communication path/link 151. Network 150 can be further communicatively coupled to remote terminal 170 via communication path/link 152 and/or trusted computer system 180 via communication path/link 153. Alternately, sensor 104 can communicate directly with remote terminal 170 and/or trusted computer systems 180 without an intervening reader device 120 being present. For example, sensor 104 can communicate with remote terminal 170 and/or trusted computer system 180 through a direct communication link to network 150, according to some embodiments, as described in U.S. Patent Application Publication 2011/0213225 an incorporated herein by reference in its entirety. Any suitable electronic communication protocol can be used for each communication path or link 141, 142, 151, 152 and/or 153, such as near field communication (NFC), radio frequency identification (RFID), BLUETOOTH® or BLUETOOTH® Low Energy protocols, WiFi, mobile telephone network, or the like. Remote terminal 170 and/or trusted computer system 180 can be accessible, according to some embodiments, by a party other than a primary user who have an interest in the primary user's lactate concentrations or rate of lactate clearance, such as the individual's trainer or coach. Reader device 120 can comprise display 122 and optional input component 121. Display 122 can comprise a touch-screen interface, according to some embodiments.

In some embodiments, sensor control device 102 can include sensor 104 that, while positioned in vivo, makes contact with the bodily fluid of the user and senses the analyte levels contained therein. The sensor can be part of the sensor control device that resides on the body of the user and contains the electronics and power supply that enable and control the analyte sensing. Sensor control device 102, and variations thereof, can also be referred to as a “sensor control unit,” an “on-body electronics” device or unit, an “on-body” device or unit, or a “sensor data communication” device or unit. In some embodiments, sensor control device 102 includes sensor housing 103, which can house circuitry and a power source for operating sensor 104. Optionally, the power source and/or active circuitry can be omitted. A processor (not shown in FIG. 2A) can be communicatively coupled to sensor 104, with the processor being physically located within sensor housing 103 or reader device 120. In some embodiments, sensor 104 protrudes from the underside of sensor housing 103 and extends through adhesive layer 105, which is adapted for adhering sensor housing 103 to a tissue surface, such as skin, according to some embodiments.

FIG. 2B shows a block diagram of a processing electronics that can be associated with one or more components of the sensing system, such as within reader device 120. Alternately, such functionality can be associated with one or more of network 150, remote terminal 170 or trusted computer system 180. As shown, processing electronics 190 receive, either directly or indirectly, a signal originating from sensor control device 102. The signal can be processed using algorithms associated with processor 191 and/or memory 192. Lactate concentrations determined therewith can be stored in memory 192 and/or exported to output device 193, which can be a display or external storage medium in various embodiments. Guidance, recommendations, and the like can also be determined using processor 191 and exported to output device 193 as well, as described further herein.

Sensor 104 is adapted to be at least partially inserted into a tissue of interest, such as within the dermal layer of the skin or in subcutaneous tissue. Sensor 104 can comprise a sensor of sufficient length for insertion to a desired depth in a given tissue. In some embodiments, the sensor can comprise a proximal portion configured to be positioned above a user's skin and a distal portion configured to be transcutaneously positioned through the user's skin and in contact with a bodily fluid. In some embodiments, the distal portion is configured to detect an analyte in the bodily fluid. In some embodiments, the proximal portion can be electrically coupled with processing electronics. In some embodiments, the processing electronics are disposed in the electronics housing of the sensor control device. The sensor can comprise a sensing region or sensing area that is active for sensing lactate, and can comprise a lactate-responsive enzyme, according to one or more embodiments. The sensing region or sensing area can include a polymeric material to which the lactate-responsive enzyme is covalently bonded, according to some embodiments. In some embodiments of the present disclosure, lactate can be monitored in any biological fluid of interest such as dermal fluid, plasma, blood, lymph, synovial fluid, cerebrospinal fluid, saliva, bronchoalveolar lavage, amniotic fluid, or the like. In some embodiments, lactate-responsive sensors of the present disclosure can be adapted for interrogating dermal fluid or interstitial fluid.

An introducer can be present transiently to promote introduction of sensor 104 into a tissue. In illustrative embodiments, the introducer can comprise a needle. It is to be recognized that other types of introducers, such as sheaths or blades, can be present in alternative embodiments. More specifically, the needle or similar introducer can transiently reside in proximity to sensor 104 prior to insertion and then be withdrawn afterward. While present, the needle or other introducer can facilitate insertion of sensor 104 into a tissue by opening an access pathway for sensor 104 to follow. For example, the needle can facilitate penetration of the epidermis as an access pathway to the dermis to allow implantation of sensor 104 to take place, according to some embodiments. After opening the access pathway, the needle or other introducer can be withdrawn so that it does not represent a sharps hazard. In some embodiments, the needle can be solid or hollow, beveled or non-beveled, and/or circular or non-circular in cross-section. In some embodiments, the needle can be comparable in cross-sectional diameter and/or tip design to an acupuncture needle, which can have a cross-sectional diameter of about 250 microns, for example. It is to be recognized, however, that suitable needles can have a larger or smaller cross-sectional diameter if needed for particular applications.

In some embodiments, a tip of the needle can be angled over the terminus of sensor 104, such that the needle penetrates a tissue first and opens an access pathway for sensor 104. In some embodiments, sensor 104 can reside within a lumen or groove of the needle, with the needle similarly opening an access pathway for sensor 104. In either case, the needle is subsequently withdrawn after facilitating insertion.

Sensor 104 can employ a two-electrode or a three-electrode detection motif, according to some embodiments of the present disclosure. Three-electrode motifs can comprise a working electrode, a counter electrode, and a reference electrode. Two-electrode motifs can comprise a working electrode and a second electrode, in which the second electrode functions as both a counter electrode and a reference electrode (i.e., a counter/reference electrode). In both two-electrode and three-electrode detection motifs, the sensing region or sensing area of sensor 104 can be in contact with the working electrode. In some embodiments, the electrodes can be at least partially stacked upon one another, as described in further detail hereinafter. In some embodiments, the various electrodes can be spaced apart from one another upon the insertion tail of sensor 104.

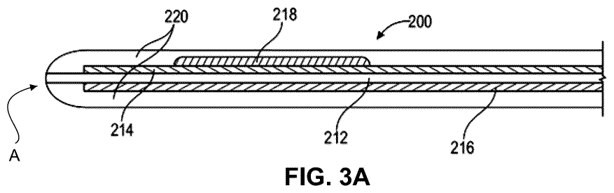

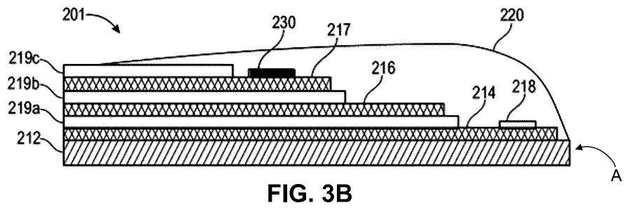

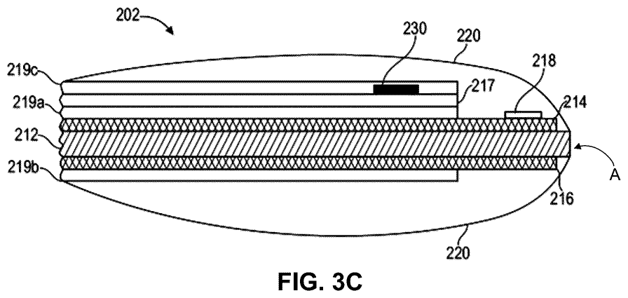

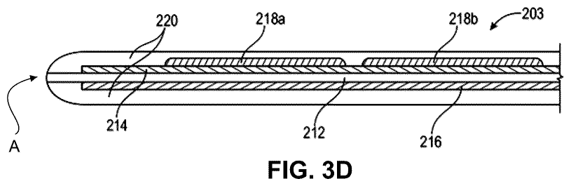

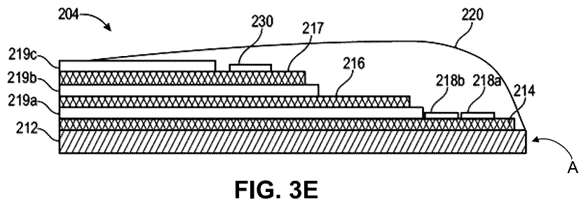

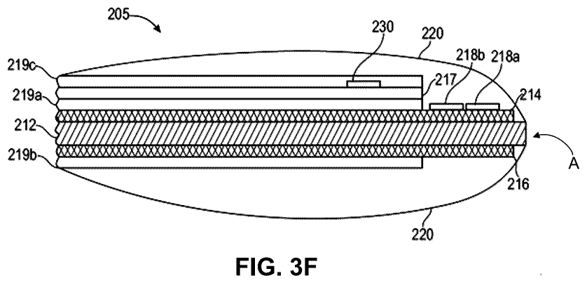

In some embodiments, the sensor can comprise a sensing area (e.g., a lactate-responsive sensing area). In some embodiments, the sensor can comprise sensing areas of different types (e.g., a glucose-responsive sensing area and lactate-responsive sensing area) upon a single working electrode or upon two or more separate working electrodes. Single working electrode sensor configurations can employ two-electrode or three-electrode detection motifs, according to some embodiments of the present disclosure and as described further herein. FIGS. 3A and 3D show cross-sectional diagrams of illustrative two-electrode analyte sensor configurations having a single working electrode, which is compatible for use in some embodiments of the disclosure herein. As shown, analyte sensors 200 and 203 comprise substrate 212 disposed between working electrode 214 and counter/reference electrode 216. Alternately, working electrode 214 and counter/reference electrode 216 can be located upon the same side of substrate 212 with a dielectric material interposed in between (configuration not shown). In some embodiments, sensor 200 includes sensing area 218 (i.e., a lactate-responsive sensing area) that can be disposed upon the surface of working electrode 214. In some embodiments, sensor 203 includes sensing areas 218a and 218b (i.e., a glucose-responsive sensing area and a lactate-responsive sensing area) that are laterally spaced apart from one another upon the surface of working electrode 214. In some embodiments, sensing area 218a can be continuously disposed or discontinuously disposed on the working electrode for detection of an analyte. In some embodiments, sensing area 218b can be continuously disposed or discontinuously disposed on the working electrode for detection of an analyte. Analyte sensor 200 can be operable for assaying lactate by any of coulometric, amperometric, voltammetric, or potentiometric electrochemical detection techniques. Analyte sensor 203 can be operable for assaying glucose and lactate by any of coulometric, amperometric, voltammetric, or potentiometric electrochemical detection techniques.

When a single working electrode is present in an analyte sensor, three-electrode sensor configurations can comprise a working electrode, a counter electrode, and a reference electrode. Related two-electrode sensor configurations can comprise a working electrode and a second electrode, wherein the second electrode can function as both a counter electrode and a reference electrode (i.e., a counter/reference electrode). In both two-electrode and three-electrode sensor configurations, both the first analyte-responsive sensing area and the second analyte-responsive sensing area can be disposed upon the single working electrode. In any of the sensor configurations disclosed herein, the various electrodes can be at least partially stacked (layered) upon one another and/or laterally spaced apart from one another upon the sensor. Suitable sensor configurations can be substantially flat in shape or substantially cylindrical in shape, with the first analyte-responsive sensing area and the second analyte-responsive sensing area being laterally spaced apart upon the working electrode. In all of the sensor configurations disclosed herein, the various electrodes can be electrically isolated from one another by a dielectric material or similar insulator.

In some embodiments, the sensor can comprise two or more working electrodes and at least one additional electrode (i.e., at least one additional electrode, at least two additional electrodes, etc.). When one additional electrode is present, the one additional electrode can function as a counter/reference electrode for each of the working electrodes. When two additional electrodes are present, one of the additional electrodes can function as a counter electrode for each of the working electrodes and the other additional electrode can function as a reference electrode for each of the working electrodes. FIGS. 3B-3C and 3E-3F show diagrams of illustrative three-electrode analyte sensor configurations, which are also compatible for use in some embodiments of the disclosure herein. Three-electrode analyte sensor configurations can be similar to that shown for analyte sensor 200 in FIGS. 3A and 3D, except for the inclusion of additional electrode 217 in analyte sensors 201, 202, 204, and 205 (FIGS. 3B-3C and 3E-3F). With additional electrode 217, counter/reference electrode 216 may then function as either a counter electrode or a reference electrode, and additional electrode 217 fulfills the other electrode function not otherwise accounted for. Working electrode 214 continues to fulfill its original function. Additional electrode 217 can be disposed upon either working electrode 214 or electrode 216, with a separating layer of dielectric material in between. For example, as depicted in FIG. 3B, dielectric layers 219a, 219b and 219c separate electrodes 214, 216, and 217 from one another and provide electrical isolation. Alternately, at least one of electrodes 214, 216, and 217 can be located upon opposite faces of substrate 212, as shown in FIG. 3C. Thus, in some embodiments, electrode 214 (working electrode) and electrode 216 (counter electrode) can be located upon opposite faces of substrate 212, with electrode 217 (reference electrode) being located upon one of electrodes 214 or 216 and spaced apart therefrom with a dielectric material. Reference material layer 230 (e.g., Ag/AgCl) can be present upon electrode 217, with the location of reference material layer 230 not being limited to the locations depicted in FIGS. 3B and 3C. As with sensor 200 shown in FIG. 3A, sensing area 218 in analyte sensors 201 and 202 can be continuously disposed or discontinuously disposed on the working electrode for detection lactate. Additionally, analyte sensors 201 and 204 can be operable for assaying lactate by any of coulometric, amperometric, voltammetric, or potentiometric electrochemical detection techniques.

Like analyte sensor 200, membrane 220 can also overcoat sensing area 218 and sensing areas 218a, 218b, as well as other sensor components, in analyte sensors 201, 202, 204, 205, thereby serving as a mass transport limiting membrane. Additional electrode 217 can be overcoated with membrane 220 in some embodiments. Although FIGS. 3B-3C and 3E-3F have depicted all of electrodes 214, 216, and 217 as being overcoated with membrane 220, it is to be recognized that only working electrode 214 may be overcoated in some embodiments. Moreover, the thickness of membrane 220 at each of electrodes 214, 216, and 217 may be the same or different. As in two-electrode analyte sensor configurations (FIGS. 3A and 3D), one or both faces of analyte sensors 201, 202, 204, 205 can be overcoated with membrane 220 in the sensor configurations of FIGS. 3B-3C and 3E-3F, or the entirety of analyte sensors 201, 202, 204, 205 can be overcoated.

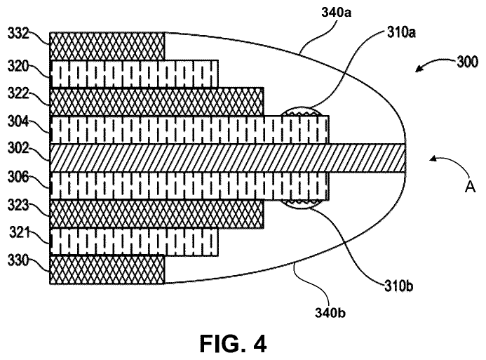

FIG. 4 shows a cross-sectional diagram of an illustrative analyte sensor configuration having two working electrodes, a reference electrode and a counter electrode, which is compatible for use in some embodiments of the disclosure herein. As shown in FIG. 4, analyte sensor 300 includes working electrodes 304 and 306 disposed upon opposite faces of substrate 302. Sensing area 310a is disposed upon the surface of working electrode 304, and sensing area 310b is disposed upon the surface of working electrode 306. Analyte sensor 300 can comprise additional sensing areas, which can be laterally spaced apart from one another upon the surface(s) of working electrode(s) 304 and/or 306. Sensing areas 310a and 310b can be lactate-responsive and glucose-responsive sensing areas, respectively, according to some embodiments of the present disclosure. Counter electrode 320 is electrically isolated from working electrode 304 by dielectric layer 322, and reference electrode 321 is electrically isolated from working electrode 306 by dielectric layer 323. Outer dielectric layers 330 and 332 are positioned upon reference electrode 321 and counter electrode 320, respectively. The layers present in the sensor 300 of FIG. 4 can comprise outer dielectric layer 332, counter electrode 320, dielectric layer 322, working electrode 304, substrate 302, working electrode 306, dielectric layer 323, reference electrode 321 and outer dielectric layer 330, in that order. The layers can be (partially) stacked on one another, with a part of each layer proximate an edge of said layer not covered by an adjacent layer. For example, substrate 302 can extend beyond (not be covered by) respective ends of working electrodes 304, 306. Working electrodes 304, 306 can extend beyond (not be covered by) the ends of dielectric layers 322, 333. Portions of the working electrodes 304, 306 that extend beyond the ends of the dielectric layers 322, 333 can provide a space for the sensing areas 310a, 310b, respectively. In any of the embodiments described herein, substrate 302 can extend to a distal tip of the sensor, so as to separate the layers on either side of the substrate 302. Membrane 340 has first membrane portion 340a and second membrane portion 340b, which separately overcoat at least sensing areas 310a and 310b, respectively, according to various embodiments, with other components of analyte sensor 300 or the entirety of analyte sensor 300 optionally being overcoated with first membrane portion 340a and/or second membrane portion 340b as well. Again, membrane 340 can be continuous but vary compositionally within first membrane portion 340a and second membrane portion 340b (i.e., upon sensing areas 310a and 310b) in order to afford different permeability values for differentially regulating the analyte flux at each location. For example, different membrane formulations can be sprayed and/or printed onto the opposing faces of analyte sensor 300. Dip coating techniques can also be appropriate, particularly for depositing at least a portion of a bilayer membrane upon one of sensing areas 310a and 310b. Accordingly, one of first membrane portion 340a and second membrane portion 340b can comprise a bilayer membrane and the other of first membrane portion 340a and second membrane portion 340b can comprise a single membrane polymer, according to some embodiments of the present disclosure. In some embodiments, analyte sensor 300 can be operable for assaying glucose and lactate by any of coulometric, amperometric, voltammetric, or potentiometric electrochemical detection techniques. The above description of the membrane applies equally to any of the sensor embodiments described herein.

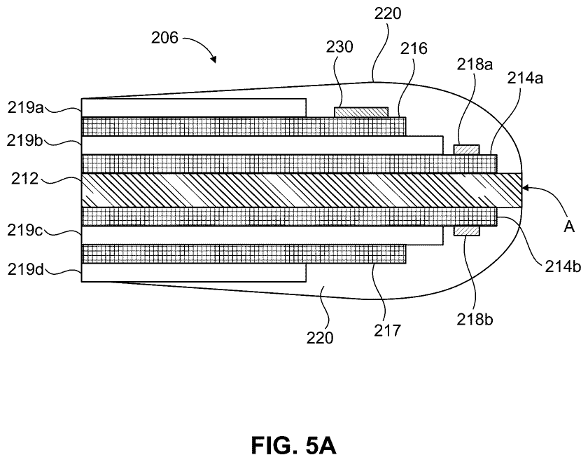

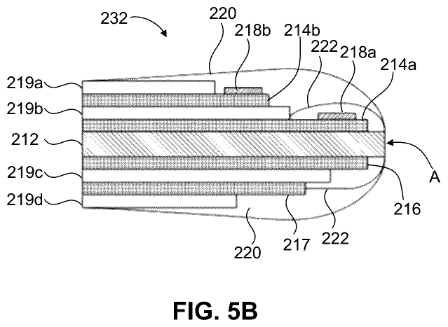

FIG. 5A shows a diagram of an illustrative four-electrode analyte sensor configuration, which is compatible for use in the disclosure herein. As shown, sensor 206 comprises substrate 212 disposed between working electrodes 214a and 214b. Alternately, working electrodes 214a and 214b can be located on the same side of substrate 212 with a dielectric material interposed in between (configuration not shown). Analyte-specific responsive sensing areas 218a and/or 218b (e.g., a glucose-responsive sensing area and a lactate-responsive sensing area) can be disposed as at least one layer upon at least a portion of working electrodes 214a and/or 214b. The analyte-responsive sensing area(s) can be continuously disposed or discontinuously disposed on the working electrode for detection of the analyte, as discussed further herein. A reference electrode can be disposed upon either working electrodes 214a or 214b, with a separating layer of dielectric material in between. A counter electrode can be disposed on the other side of working electrodes 214a or 214b, with a separating layer of dielectric material in between. For example, as depicted in FIG. 5A, dielectric layers 219b and 219c separate electrodes 214a, 214b, 216, and 217 from one another and provide electrical isolation. Outer dielectric layers 219a and 219d are positioned on reference electrode 216 and counter electrode 217. In other embodiments, at least one of electrodes 214a, 214b, 216, and 217 can be located upon opposite faces of substrate 212 (configuration not shown).

In some embodiments, electrode 214a (working electrode) and electrode 216 (counter electrode) can be located upon opposite faces of substrate 212 as electrode 217 (reference electrode), with working electrode 214b being located on the opposite face of the substrate, as depicted in FIG. 5A. Reference material layer 230 (e.g., Ag/AgCl) can be present upon reference electrode 216, with the location of reference material layer 230 not being limited to that depicted in FIG. 5A. Additionally, analyte sensor 206 can be operable for assaying the analyte by any of coulometric, amperometric, voltammetric, or potentiometric electrochemical detection techniques. Although FIG. 5A has depicted all of electrodes 214a, 214b, 216, and 217 as being overcoated with membrane 220, it is to be recognized that only working electrodes 214a and 214b can be overcoated in some embodiments. Moreover, the thickness of membrane 220 at each of electrodes 214a, 214b, 216, and 217 can be the same or different, in any of the embodiments described herein. As in two-electrode analyte sensor configurations, one or both faces of analyte sensor 206 can be overcoated with membrane 220 in the sensor configurations of FIG. 5A, or the entirety of analyte sensors 206 can be overcoated. Accordingly, the multiple-electrode sensor configuration shown in FIG. 5A should be understood as being non-limiting of the embodiments disclosed herein, with alternative electrode and/or layer configurations remaining within the scope of the present disclosure.

In some embodiments (including any of the embodiments of FIGS. 3-6), the substrate is formed from any suitable inert material. In some embodiments, the substrate is biocompatible. Examples of a suitable substrate include titanium, a carbon-based substrate (e.g., cellulose, polylactic acid) and a plastic substrate (e.g., polyethylene terephthalate, polyethylene, polypropylene, polymethylmethacrylate, polysulfone, polydimethylsiloxane, polyvinyl chloride, etc.). The substrate can be disposed between the working electrode and a counter and/or reference electrode.

In some embodiments (including any of the embodiments of FIGS. 3-6), the dielectric layer can comprise a suitable dielectric material that can form a solid. In an example, the insulation layer can be formed from porcelain (ceramic), mica, glass, barium strontium titanate, a plastic (e.g., polystyrene, polytetrafluoroethylene, polyethylene terephthalate, polyethylene, polypropylene, polymethylmethacrylate, polysulfone, polydimethylsiloxane, polyvinyl chloride), or a metal oxide (e.g., silica, alumina, titania, zirconia, tantalum oxide, etc.).

In some embodiments (including any of the embodiments of FIGS. 3-6), membrane 220 optionally overcoats at least analyte-responsive sensing areas 218a and 218b and overcoats some or all of working electrodes 214a and/or 214b and/or reference electrode 216 and/or counter electrode 217, or the entirety of analyte sensor 202 according to some embodiments. One or both faces of analyte sensor 202 can be overcoated with membrane 220. Membrane 220 can comprise one or more polymeric membrane materials having capabilities of limiting analyte flux to sensing area 218 (i.e., membrane 220 is a mass transport limiting membrane having some permeability for the analyte(s) being measured). The composition and thickness of membrane 220 can vary to promote a desired analyte flux to analyte-responsive sensing areas 218a, 218b, thereby providing a desired signal intensity and stability.

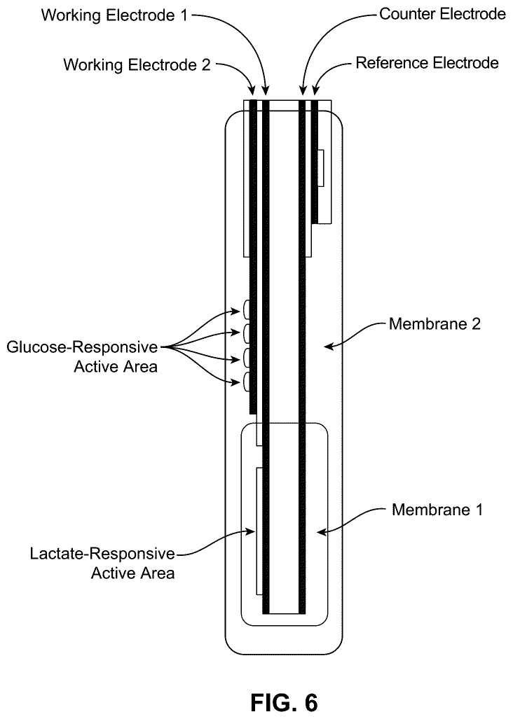

FIG. 5B and FIG. 6 show diagrams of an illustrative four-electrode analyte sensor configuration, which is compatible for use in the disclosure herein. As shown, analyte sensor 232 comprises substrate 212 disposed between working electrode 214a and counter electrode 216. Working electrodes 214a and 214b are located on the same side of substrate 212 with a dielectric material 219b interposed in between working electrodes 214a and 214b. Counter electrode 216 and reference electrode 217 are located on the opposite side of substrate 212 with a dielectric material 219c interposed in between counter electrode 216 and reference electrode 217. Analyte-specific responsive sensing area 218a (e.g., lactate-responsive sensing area) can be disposed as at least one layer upon at least a portion of working electrode 214a. Analyte-specific responsive sensing area 218b (e.g., a glucose-responsive) can be disposed as at least one layer upon at least a portion of working electrode 214b. Sensing area 218a (e.g., lactate-responsive sensing area) can be located closer to distal end A than analyte-specific responsive sensing area 218b (e.g., a glucose-responsive). The analyte-responsive sensing area(s) 218a and 218b can be continuously disposed or discontinuously disposed on the working electrode(s) for detection of the analyte, as discussed further herein. As depicted in FIG. 5B, dielectric layers 219b and 219c separate electrodes 214a, 214b, 216, and 217 from one another and provide electrical isolation. Outer dielectric layers 219a and 219d are positioned on working electrode 214b and counter electrode 217. Reference material layer 230 (e.g., Ag/AgCl) (not shown) can be present upon reference electrode 216, or another suitable location on the sensor. The layers present in the sensor 232 of FIG. 5B can comprise outer dielectric layer 219a, working electrode 214b, dielectric layer 219b, working electrode 214a, substrate 212, reference electrode 216, dielectric layer 219c, counter electrode 217 and outer dielectric layer 219d, in that order. The layers may be (partially) stacked on one another, with a part of each layer proximate an end of said layer not covered by an adjacent layer. As shown in FIG. 6, the lactate-responsive sensing area can be continuously disposed on working electrode 214a and the glucose-responsive sensing area can be discontinuously disposed on working electrode 214b. Additionally, analyte sensors 206, 232 can be operable for assaying the analytes by any of coulometric, amperometric, voltammetric, or potentiometric electrochemical detection techniques.

In some embodiments, sensor 232 can contain two membranes 220, 222. As seen in FIG. 5B, membrane 222 can only cover a portion of working electrode 214a, which includes sensing area 218a (e.g., lactate-responsive sensing area). Membrane 220 can cover both sensing area 218a (e.g., lactate-responsive sensing area) and sensing area 218b (e.g., a glucose-responsive). Membrane 220 can also cover counter electrode 216 and reference electrode 217 on the opposite side of substrate 212. Thus, sensing area 218a (e.g., lactate-responsive sensing area) can have a bilayer membrane that includes membranes 222 and 220, while sensing area 218b can only have a single layer membrane 220. Although FIG. 5B has depicted all of electrodes 214a, 214b, 216, and 217 as being overcoated with membrane 220, it is to be recognized that only working electrodes 214a and 214b can be overcoated in some embodiments. Moreover, the thickness of membranes 220, 222 at each of electrodes 214a, 214b, 216 and 217 can be the same or different. As in two-electrode analyte sensor configurations, one or both faces of analyte sensor 232 can be overcoated with membrane 220 in the sensor configurations of FIG. 5B, or the entirety of analyte sensor 232 can be overcoated. Accordingly, the multiple-electrode sensor configuration shown in FIGS. 5A and 5B should be understood as being non-limiting of the embodiments disclosed herein, with alternative electrode and/or layer configurations remaining within the scope of the present disclosure.

Membrane 222 can be dip coated onto sensing area 218a (e.g., lactate-responsive sensing area). For example, sensor 232 can be partially dipped into a membrane solution such that only an end region near distal end A, which includes sensing area 218a and does not include sensing area 218b, is submerged into the membrane solution. The application of membrane 222 can be accomplished in a single dip procedure or can require multiple dips into the membrane solution to obtain a dense membrane. A larger portion of sensor 232, which includes both sensing areas 218a and 218b, can then be submerged into a different membrane solution. Thus, sensing area 218a, which is located closer to a distal end A, can have a bilayer membrane, while sensing area 218b, which is proximal relative to sensing area 218a, would have a single layer membrane. Dip coating in this manner has numerous advantages. First, dispensing both sensing areas on one side of substrate 212 without needing to flip the substrate 212 simplifies the manufacturing process and improves efficiency. Second, this dipping method allows for use of the same membrane dipping equipment to be used for both membranes 222, 220, by simply exchanging out the membrane solutions and adjusting dipping depth.

In some embodiments, membrane 222 can comprise at least a crosslinked polyvinylpyridine homopolymer or copolymer. Membrane 222 can be single-component or multi-component. Multi-component membrane embodiments can comprise a bilayer or homogeneous admixture of the crosslinked polyvinylpyridine and another polymer, according to some embodiments of the present disclosure. Suitable polyvinylpyridine copolymers for inclusion in membrane 222 can comprise up to about 25% comonomers (based on the total amount of monomers in the copolymer), such as from about 0.1% to about 5% comonomers, or about 5% to about 15% comonomers, or about 15% to about 25% comonomers, or about 1% to about 10% co-monomers. Suitable comonomers are not particularly limited, provided that the mass transport limiting membrane affords sufficient lactate permeability to provide an analyte sensitivity of about 1 nA/mM or greater when exposed to lactate. In some embodiments, the polyvinylpyridine copolymer can be distinct from a polyvinylpyridine-co-styrene copolymer, according to some embodiments. Crosslinking of the membrane polymers disclosed herein for membrane 222 can take place through functionalization with a bis-epoxide, such as polyethylene glycol diglycidyl ether (PEGDGE) or glycerol triglycidyl ether. In some embodiments, membrane 222 can comprise polyvinylpyridine and a crosslinker, such as polyethylene glycol diglycidyl ether (PEGDGE), e.g., PEGDGE 400.

In some embodiments, membrane 220 can be a membrane comprising crosslinked polymers containing heterocyclic nitrogen groups, such as polymers of polyvinylpyridine and polyvinylimidazole. In some embodiments, membrane 220 can comprise polyurethane, or polyether urethane, or chemically related material, or membranes that are made of silicone, and the like.

In some embodiments, a membrane can be formed by crosslinking in situ a polymer, including those discussed above, modified with a zwitterionic moiety, a non-pyridine copolymer component, and optionally another moiety that is either hydrophilic or hydrophobic, and/or has other desirable properties, in an buffer solution (e.g., an alcohol-buffer solution). In some embodiments, the modified polymer can be made from a precursor polymer containing heterocyclic nitrogen groups. For example, a precursor polymer can be polyvinylpyridine or polyvinylimidazole. Optionally, hydrophilic or hydrophobic modifiers can be used to “fine-tune” the permeability of the resulting membrane to an analyte of interest. Optional hydrophilic modifiers, such as poly(ethylene glycol), hydroxyl or polyhydroxyl modifiers, and the like, and any combinations thereof, can be used to enhance the biocompatibility of the polymer or the resulting membrane.

In some embodiments, membrane 220 can comprise a polymer including, but not limited to, poly(styrene-co-maleic anhydride), dodecylamine and poly(propylene glycol)-block-poly(ethylene glycol)-block-poly(propylene glycol) (2-aminopropyl ether) crosslinked with poly(propylene glycol)-block-poly(ethylene glycol)-block-poly(propylene glycol) bis(2-aminopropyl ether); poly(N-isopropyl acrylamide); a copolymer of poly(ethylene oxide) and poly(propylene oxide); polyvinylpyridine; a derivative of polyvinylpyridine; polyvinylimidazole; a derivative of polyvinylimidazole; and the like; and any combination thereof. In some embodiments, the membrane can comprise a polyvinylpyridine-co-styrene polymer, in which a portion of the pyridine nitrogen atoms are functionalized with a non-crosslinked poly(ethylene glycol) tail and a portion of the pyridine nitrogen atoms are functionalized with an alkylsulfonic acid group. In some embodiments, membrane 220 can comprise the polymer shown below.

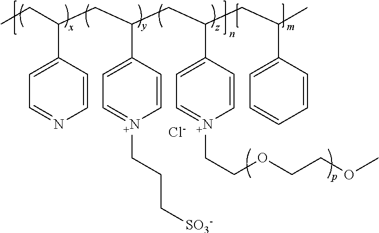

In some embodiments, membrane 222 can comprise polyvinylpyridine and a crosslinker, such as polyethylene glycol diglycidyl ether (PEGDGE), e.g., PEGDGE 400. In some embodiments, membrane 220 can comprise polyvinylpyridine-co-styrene and a crosslinker, such as PEGDGE, e.g., PEGDGE 400, or glycerol triglycidyl ether. In some embodiments, membrane 222 can comprise polyvinylpyridine and a crosslinker, such as PEGDGE, e.g., PEGDGE 400, and membrane 220 can comprise polyvinylpyridine-co-styrene and a crosslinker, such as PEGDGE, e.g., PEGDGE 400, or glycerol triglycidyl ether.

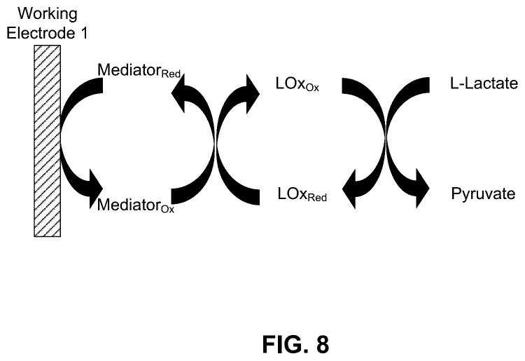

In some embodiments, sensing area 218a can comprise a lactate-responsive enzyme. More particularly, the lactate-responsive enzyme can comprise lactate dehydrogenase or lactate oxidase, according to some embodiments of the present disclosure. FIG. 8 shows a diagram of an enzyme system that can be used for detecting lactate according to the disclosure herein. In some embodiments, an electron transfer mediator can facilitate conveyance of electrons from lactate to working electrode 214a during a redox reaction as shown in FIG. 8. The electrons transferred during this reaction provide the basis for lactate detection in the working electrode. Changes in the signal intensity (e.g., current) at working electrode 214a can be proportional to the lactate concentration and/or the activity of the lactate-responsive enzyme.

In some embodiments, sensing area 218a can further comprise a stabilizer for lactate dehydrogenase or lactate oxidase, such as catalase or albumin. According to some embodiments, the lactate-responsive enzyme, such as lactate dehydrogenase or lactate oxidase, can be covalently bonded to a polymer comprising sensing region 218. Covalent bonding immobilizes the lactate-responsive enzyme in sensing area 218a.

In some embodiments, lactate oxidase can be present in the sensing area in an amount ranging from about 0.05 g to about 5 g, or from about 0.1 g to about 4 g, or from about 0.2 g to about 3 g, or from about 0.5 g to about 2 g. In terms of weight percentage of the sensing area, the lactate oxidase can be present in an amount ranging from about 10% to about 90% by weight of the sensing area, or from about 25% to about 75% by weight of the sensing area, or from about 30% to about 60% by weight of the sensing area.

According to some embodiments, the albumin within the sensing area can comprise human serum albumin. In some embodiments, non-human albumin can be satisfactorily used, such as bovine serum albumin.

The albumin can be incorporated within the sensing area in an amount sufficient to stabilize the lactate-responsive enzyme, particularly lactate oxidase, according to the disclosure herein. In more specific embodiments, the albumin can be present in the sensing area in an amount ranging from about 0.05 g to about 5 kg, or from about 0.1 g to about 2 kg, or from about 0.2 kg to about 1.5 kg, or from about 0.3 kg to about 0.8 kg. In terms of weight percentage of the sensing area, the albumin can be present in an amount ranging from about 25% to about 75% by weight of the sensing area, or from about 30% to about 60% by weight of the sensing area. In certain embodiments, the weight ratio of lactate oxidase to albumin can range from about 10:1 to about 1:10 (w/w), or from about or from about 5:1 to about 1:5, or from about 5:1 to about 1:1, or from about 2:1 to about 1:1, or from about 1:1 to about 1:5, or from about 1:1 to about 1:2. In some embodiments, the weight ratio of lactate oxidase to albumin can be about 2:1. In some embodiments, the weight ratio of lactate oxidase to albumin can be about 1:1.

In some embodiments, sensing area 218a can comprise a redox mediator that can comprise a polymer and an electron transfer agent. In some embodiments, the electron transfer agent can be a low-potential osmium complex electron transfer mediator. In some embodiments, the polymer is covalently bonded to both the lactate-responsive enzyme, such as lactate dehydrogenase or lactate oxidase, and a low-potential osmium complex electron transfer mediator, as disclosed in, for example, U.S. Pat. Nos. 6,134,461, 6,605,200, 6,736,957, 7,501,053, and 7,754,093, the disclosures of each of which are incorporated herein by reference in their entirety. Other suitable examples of electron transfer mediators and polymer-bound electron transfer mediators can include those described in U.S. Pat. Nos. 8,444,834, 8,268,143, and 6,605,201, the disclosures of which are incorporated herein by reference in their entirety.

The electron transfer mediator can facilitate conveyance of electrons from lactate to working electrode 214a during a redox reaction. Changes in the signal intensity (e.g., current) at working electrode 214 can be proportional to the lactate concentration and/or the activity of the lactate-responsive enzyme. A calibration factor can be applied (e.g., by a processor) to determine the lactate concentration from the signal intensity, according to some embodiments. Suitable electron transfer mediators include electroreducible and electrooxidizable ions, complexes or molecules having redox potentials that are a few hundred millivolts above or below the redox potential of the standard calomel electrode (SCE). Other suitable electron transfer mediators can comprise metal compounds or complexes of ruthenium, iron (e.g., polyvinylferrocene), or cobalt, for example. Suitable ligands for the metal complexes can include, for example, bidentate or higher denticity ligands such as, for example, a bipyridine, biimidazole, phenanthroline, or pyridyl(imidazole). Other suitable bidentate ligands can include, for example, amino acids, oxalic acid, acetylacetone, diaminoalkanes, or o-diaminoarenes. Any combination of monodentate, bidentate, tridentate, tetradentate, or higher denticity ligands can be present in the metal complex to achieve a full coordination sphere.

The polymer in the redox mediator can be any suitable polymer that allows the transfer of electrons between the electron transfer agent and the working electrode. For example, the polymer can be, poly(4-vinylpyridine), poly(1-vinylimidazole), poly(thiophene), poly(aniline), poly(pyrrole), poly(acetylene), poly(acrylic acid), styrene/maleic anhydride copolymer, methylvinylether/maleic anhydride copolymer, poly(vinylbenzylchloride), poly(allylamine), poly(lysine), poly(acrylamide-co-1-vinyl imidazole), poly(4-vinylpyridine) quaternized with carboxypentyl groups, and poly(sodium 4-styrene sulfonate). These polymers can be considered precursor polymers in that the polymers are further modified to immobilize (e.g., attach) the electron transfer complex. In some embodiments, the polymer can comprise a poly(4-vinylpyridine), poly(1-vinylimidazole), poly(thiophene), poly(aniline), poly(pyrrole), or poly(acetylene) backbone. In other embodiments, the polymer can comprise a polymer or copolymer repeat unit that can comprise at least one (e.g., 1, 2, 3, 4, 5, or 6) pendant pyridinyl group, imidazolyl group, or both a pyridinyl and imidazolyl group. For example, a suitable polymer includes partially or fully quaternized poly(4-vinylpyridine) and poly(1-vinylimidazole) in which quaternized pyridine and imidazole groups, respectively, can be used to form spacers by reaction with (e.g., complexation with) an electron transfer agent.

Suitable polymers for inclusion in sensing area 218a include, but are not limited to, polyvinylpyridines (e.g., poly(4-vinylpyridine)), polyimidazoles (e.g., poly(1-vinylimidazole), or any copolymer thereof. Illustrative copolymers that can be suitable include, for example, copolymers containing monomer units such as styrene, acrylamide, methacrylamide, or acrylonitrile.

Covalent bonding of the lactate-responsive enzyme to a polymer or other matrix (e.g., sol-gel) in sensing area 218a can take place via a crosslinker introduced with a suitable crosslinking agent. Suitable crosslinking agents for reaction with free amino groups in the enzyme (e.g., with the free amine in lysine) can include crosslinking agents such as, for example, polyethylene glycol diglycidylether (PEGDGE) or other polyepoxides, cyanuric chloride, N-hydroxysuccinimide, imidoesters, epichlorohydrin, or derivatized variants thereof. Suitable crosslinking agents for reaction with free carboxylic acid groups in the enzyme can include, for example, carbodiimides.

In some embodiments, the redox mediator can comprise an osmium complex bonded to a polymer or copolymer of poly(1-vinyl imidazole) or poly(4-vinylpyridine). The poly(4-vinylpyridine)-based polymer is a prepolymer that has been modified, as shown in the following structure, to attach an osmium complex (e.g., a poly(biimidizyl) osmium complex).

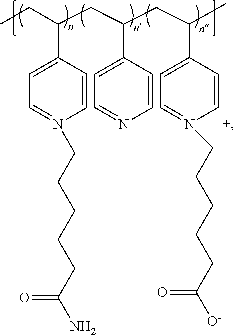

wherein n can be 2, n′ can be 17, and n″ can be 1. Other reactive groups and/or spacer groups can be used.

In some embodiments, the electron redox mediator can comprise an osmium-containing poly(4-vinylpyridine)-based polymer, as shown below.

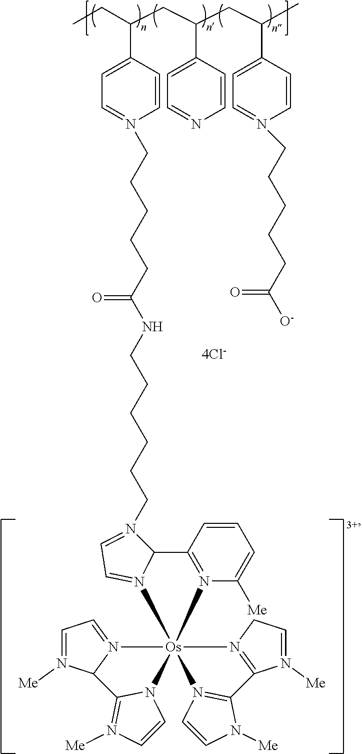

wherein n is 2, n′ is 17, and n″ is 1.

Although the lactate-responsive enzyme and/or the electron transfer mediator can be covalently bonded to a polymer or other suitable matrix in sensing area 218a, other association means can be suitable as well. In some embodiments, the lactate-responsive enzyme and/or the electron transfer mediator can be ionically or coordinatively associated with the polymer or other matrix. For example, a charged polymer can be ionically associated with an oppositely charged lactate-responsive enzyme or electron transfer mediator. In still other embodiments, the lactate-responsive enzyme and/or the electron transfer mediator can be physically entrained within the polymer or other matrix of sensing area 218a.

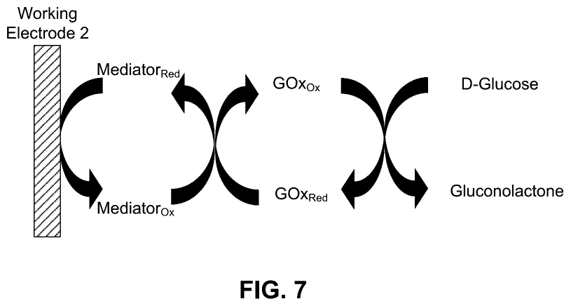

In some embodiments, sensing area 218b can comprise a redox mediator that can comprise a polymer and an electron transfer agent. Suitable polymers and electron transfer agents discussed with respect to the first sensing area 218a are also suitable for the second sensing area 218b. In some embodiments, the electron transfer agent can be a low-potential osmium complex electron transfer mediator. In some embodiments, the polymer is covalently bonded to both the glucose-responsive enzyme, such as glucose oxidase, and the low-potential osmium complex electron transfer mediator. FIG. 7 shows a diagram of an enzyme system that can be used for detecting glucose according to the disclosure herein. The electron transfer mediator can facilitate conveyance of electrons from glucose to working electrode 214b during a redox reaction. Changes in the signal intensity (e.g., current) at working electrode 214b can be proportional to the glucose concentration and/or the activity of the glucose-responsive enzyme. A calibration factor can be applied (e.g., by a processor) to determine the glucose concentration from the signal intensity, according to some embodiments.

In some embodiments, the first sensing area 218a can comprise a lactate-responsive enzyme, such as lactate oxidase, and the second sensing area 218b can comprise a glucose-responsive enzyme, such as glucose oxidase, in addition to the suitable electron transfer agents and polymers discussed in more detail above. According to some embodiments, the sensor can be suitable for detecting glucose and lactate and can comprise a first working electrode 214a having a first sensing area 218a disposed thereon and a second working electrode having a second sensing area 218b disposed thereon, and a mass transport limiting membrane overcoating the first and second sensing areas upon the working electrode, in which the first sensing area 218a comprises a polymer, an albumin, and a lactate-responsive enzyme (e.g., lactate oxidase) covalently bonded to the polymer and the second sensing area 218b comprises a glucose-responsive enzyme (e.g., glucose oxidase) covalently bonded to a polymer. In some embodiments, the first and second electron transfer agents can be different from one another. In some embodiments, the mass transport limiting membrane can comprise at least a crosslinked polyvinylpyridine homopolymer or copolymer. The composition of the mass transport limiting membrane can be the same or different where the mass transport limiting membrane overcoats each sensing area. In some embodiments, the mass transport limiting membrane overcoating the second sensing area can be single-component (contain a single membrane polymer) and the mass transport limiting membrane overcoating the first sensing area can be multi-component (contain two or more different membrane polymers, one of which is a polyvinylpyridine homopolymer or copolymer), either as a bilayer or homogeneous admixture.

In some embodiments, the sensor discussed herein can be configured to be partially inserted into an individual's skin. The working electrodes are disposed upon the sensor and can be inserted in the tissue to facilitate lactate and glucose analyses therein. Suitable tissues are not considered to be particularly limited and specific examples are addressed in more detail above. Similarly, considerations for deploying a sensor at a particular position or depth within a tissue are addressed above.

Detection methods for assaying glucose and lactate employing the sensor disclosed herein can comprise: exposing an analyte sensor to a fluid comprising glucose and lactate; applying a potential to the first working electrode and the second working electrode, obtaining a first signal at or above an oxidation-reduction potential of the lactate-responsive sensing area, in which the first signal is proportional to a concentration of lactate in the fluid, obtaining a second signal at or above an oxidation-reduction potential of the glucose-responsive sensing area, in which the second signal is proportional to a concentration of glucose in the fluid, and correlating the first signal to the concentration of lactate in the fluid and the second signal to the concentration of glucose in the fluid.

In some embodiments, the signals associated with each sensing area can be correlated to a corresponding concentration of glucose or lactate by consulting a lookup table or calibration curve for each analyte. A lookup table for each analyte can be populated by assaying multiple samples having known analyte concentrations and recording the sensor response at each concentration for each analyte. Similarly, a calibration curve for each analyte can be determined by plotting the analyte sensor response for each analyte as a function of the concentration and determining a suitable calibration function over the calibration range (e.g., by regression, particularly linear regression).

A processor can determine which sensor response value in a lookup table is closest to that measured for a sample having an unknown analyte concentration and then report the analyte concentration accordingly. In some embodiments, if the sensor response value for a sample having an unknown analyte concentration is between the recorded values in the lookup table, the processor can interpolate between two lookup table values to estimate the analyte concentration. Interpolation can assume a linear concentration variation between the two values reported in the lookup table. Interpolation can be employed when the sensor response differs a sufficient amount from a given value in the lookup table, such as variation of about 10% or greater.

Likewise, according to some embodiments, a processor can input the sensor response value for a sample having an unknown analyte concentration into a corresponding calibration function. The processor can then report the analyte concentration accordingly.

In some embodiments, the sensors described herein can also be configured to analyze for other analytes as well. Additional analytes that can be of interest in the sports training realm include, for example, markers of cardiac stress, markers of inflammation, pyruvate, pH, triglycerides, free fatty acids, and hormones such as insulin, glucagon, cortisol, epinephrine, norepinephrine, testosterone, HGH, IFG1, and BDNF.

In some embodiments, the sensing system can incorporate further functionality appropriate for monitoring physical activity. Additional functionality that can optionally be present include, for example, a heart rate monitor, a heart rate variability monitor, a blood oxygen monitor, a power meter, an accelerometer, a pedometer, a measure of rate of perceived exertion by user, or the like.

It is to be appreciated that the sensing system and sensor disclosed herein can comprise additional features and/or functionality that are not necessarily described herein in the interest of brevity. Thus, the foregoing description of the sensing system and sensor should be considered illustrative and non-limiting in nature.

### Methods

Also disclosed herein are a method for monitoring lactate levels in an individual and methods for determining an aerobic threshold and an anaerobic threshold in the individual. The methods disclosed herein allow for a quick and convenient way to obtain reliable lactate levels correlated to a metric like power and heart rate, despite a lag in lactate concentration change in the interstitial fluid in response to a power or heart rate change, and determine lactate thresholds to provide an individual with valuable information about a running pace, heart rate, and/or power that corresponds to an aerobic or anaerobic threshold.

In some embodiments, the method for monitoring lactate levels in an individual comprises exposing an analyte sensor of a sensing system to a fluid. In some embodiments, the sensor can be any sensor as disclosed herein. In some embodiments, the sensor can be a sensor as disclosed in U.S. Pat. No. 10,392,647, US 2019/0320947, and/or US 2022/0125354, the disclosures of each of which are incorporated herein by reference in their entirety. In some embodiments, the analyte sensor is the sensor described in relation to FIGS. 3-6. In some embodiments, the analyte sensor is the sensor described in relation to FIGS. 5B and 6. In some embodiments, the method further comprises: applying a potential to a working electrode of the sensor; obtaining a first signal at or above an oxidation-reduction potential of a lactate-responsive sensing area (e.g., a first sensing area), the signal being proportional to a concentration of lactate in the fluid; and correlating the signal to the concentration of lactate in the fluid. In some embodiments, the method further comprises obtaining a second signal at or above an oxidation-reduction potential of the glucose-responsive sensing area (e.g., a second sensing area), the second signal being proportional to a concentration of glucose in the fluid; and correlating the second signal to the concentration of glucose in the fluid. In some embodiments, each sensing area has an oxidation-reduction potential, and the oxidation-reduction potential of the glucose-responsive sensing area is sufficiently separated from the oxidation-reduction potential of the lactate-responsive sensing area to allow independent production of signals from the glucose-responsive sensing area and the lactate-responsive sensing area.

In some embodiments, the method for determining an aerobic threshold, anaerobic threshold, or both the aerobic threshold and anaerobic threshold, can comprise continuously measuring signals indicative of lactate concentrations in a biological fluid in the individual in vivo with a sensing system comprising a lactate-responsive sensor. As discussed above, in some embodiments, the sensor can be any sensor as disclosed herein or a sensor disclosed in one of the publications referenced above. In some embodiments, measuring the plurality of lactate concentrations is conducted when the individual is performing a lactate threshold test to determine an aerobic threshold and anaerobic threshold. In some embodiments, the lactate threshold test can be a step test with incremental increases in exercise intensity. In some embodiments, the lactate threshold test can comprise running at a lower speed (e.g., on a treadmill) and then running incrementally faster until exhaustion or near exhaustion. In some embodiments, the lactate threshold test can comprise biking (e.g., on a stationary bike) at a lower speed and then biking incrementally faster until exhaustion or near exhaustion. In some embodiments, the incremental increases can be defined based on power. In some embodiments, the incremental increases can be about 1 w, about 2 w, about 3 w, about 4, w about 5 w, about 6 w, about 7w, about 8 w, about 9 w, about 10 w, about 15 w, about 20 w, about 25 w, about 30 w, about 35 w, about 40 w, about 45 w, or about 50 w. In some embodiments, the lactate threshold test can collect lactate concentration data relative to power or heart rate.

In some embodiments, the sensor is configured to take a measurement about every second, about every 3 seconds, about every 5 seconds, about every 10 seconds, about every 15 seconds, about every 20 seconds, about every 30 seconds, about every 45 seconds, about every minute, about every 1.5 minutes, about every 2 minutes, about every 3 minutes, about every 5 minutes, about every 10 minutes, about every 15 minutes, about every 20 minutes, about every 30 minutes, about every 45 minutes, about every hour, about every 2 hours, or about every 3 hours. In some embodiments, the method can further comprise communicating a signal indicative of the lactate concentration measured by the lactate-responsive sensor to the processor. In some embodiments, the processor can be configured to receive the signal from the lactate-responsive sensor and determine a lactate concentration from the received signal. In some embodiments, the processor is further configured to determine an aerobic threshold, an anaerobic threshold, or both the aerobic threshold and anaerobic threshold, of the individual based on the signals indicative of lactate concentrations from the lactate-responsive sensor. In some embodiments, the processor can further signal to an individual wearing the sensor or another interested party when the aerobic threshold or the anaerobic threshold has been reached. In some embodiments, the output of the processor can be numerical and/or graphical. The notification to the wearer of the lactate-responsive sensor or other interested party can be auditory, tactile (haptic), or any combination thereof

In some embodiments, the method can comprise determining a baseline concentration of lactate for the individual based on the plurality of lactate concentrations measurements. Baseline concentrations of lactate can vary from individual to individual. In some embodiments, the baseline lactate concentration can range from about 0.5 mM to about 2 mM, about 0.5 mM to about 1.5 mM, or about 0.5 mM to about 1 mM. The baseline concentration of lactate can be determined prior to reaching an anaerobic threshold and/or after the lactate concentrations have again stabilized after reaching an anaerobic threshold.

In some embodiments, an aerobic threshold can be a fixed value and defined as the power or heart rate at which an initial rise of about 0.5 mM or about 1 mM (or another predetermined suitable value) from a baseline concentration of lactate is seen. In some embodiments, the processor is configured to determine the baseline concentration from the lactate measurements and determine the aerobic threshold by adding about 0.5 mM or about 1 mM to the baseline lactate concentration such that the aerobic threshold can be defined in terms of (i) lactate concentration and (ii) power or heart rate correlated to that lactate concentration.

In some embodiments, the processor is configured to determine an aerobic threshold based on the plurality of lactate concentrations using a log-log model. In some embodiments, the log-log model can comprise plotting lactate levels against intensity (e.g., power or heart rate) on a logarithmic scale. In some embodiments, the log-log model can further comprise dividing the plot into two segments and conducting a segmented regression analysis. Each segment can have a best fit line connected at a breakpoint. In some embodiments, the segmented regression analysis can comprise minimizing the sum of the squared error (SSE), also known as the residual sum of squares (RSS), for example by varying the membership of data points in each group. In some embodiments, the breakpoint provides the aerobic threshold in terms of (i) lactate concentration and (ii) intensity (e.g., power or heart rate).

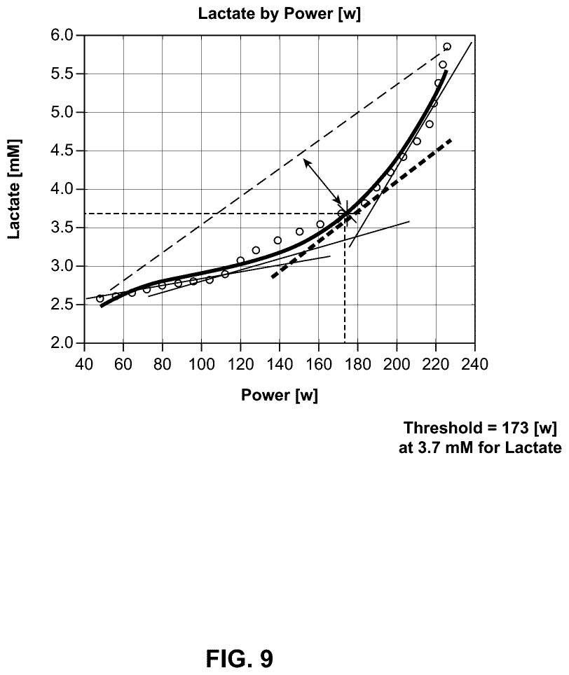

In some embodiments, an anaerobic threshold can be defined as the power or heart rate at a fixed lactate concentration of 4 mM. In some embodiments, the processor is configured to determine an anaerobic threshold based on the plurality of lactate concentrations using a polynomial regression model. In some embodiments, the processor is configured to determine an anaerobic threshold based on the plurality of lactate concentrations using a D-max method as shown in FIG. 9. In some embodiments, the D-max method can comprise conducting a regression analysis to determine a best fit line (e.g., regression curve) based on the plurality of lactate concentrations plotted against power or heart rate. In some embodiments, a 3^{rd} order polynomial function can provide the best fit line (e.g., regression curve) based on the plurality of lactate concentrations plotted against power or heart rate. In some embodiments, the D-max method can further comprise finding a linear equation that the first measured lactate concentration (e.g., start point) to the last measured lactate concentration (e.g., end point). In some embodiments, the D-max method can further include finding the D-max point on the best fit line (e.g., regression curve) that is furthest from the linear line connecting the first measured lactate concentration (e.g., start point) to the last measured lactate concentration (e.g., end point), wherein the D-max point is on a tangent line to the curve that is parallel to the linear line connecting the first measured lactate concentration (e.g., start point) to the last measured lactate concentration (e.g., end point). In some embodiments, the D-max point provides the anaerobic threshold in terms of (i) lactate concentration and (ii) power or heart rate.

In some embodiments, the processor is configured to determine an anaerobic threshold based on the plurality of lactate concentrations using a modified D-max method. In some embodiments, the modified D-max method is similar to the D-max method described above except that the start point is a lactate concentration of 0.4 mM (or another suitable start point). That is, in some embodiments, the modified D-max method can comprise finding a linear equation connecting a lactate concentration of 0.4 mM (e.g., start point) to the last measured lactate concentration (e.g., end point). In some embodiments, the modified D-max method can comprise conducting a regression analysis to determine a best fit line (e.g., regression curve) based on the plurality of lactate concentrations plotted against power or heart rate. In some embodiments, a 3^{rd} order polynomial function can provide the best fit line (e.g., regression curve) based on the plurality of lactate concentrations plotted against power or heart rate. In some embodiments, the modified D-max method further includes finding the D-max point on the fit line (e.g., regression curve) that is furthest from the linear line connecting the lactate concentration of 0.4 mM (e.g., start point) to the last measured lactate concentration (e.g., end point) and is on a tangent line to the curve that is parallel to the linear line connecting the lactate concentration of 0.4 mM (e.g., start point) to the last measured lactate concentration (e.g., end point). In some embodiments, the D-max point provides the anaerobic threshold in terms of (i) lactate concentration and (ii) power or heart rate.

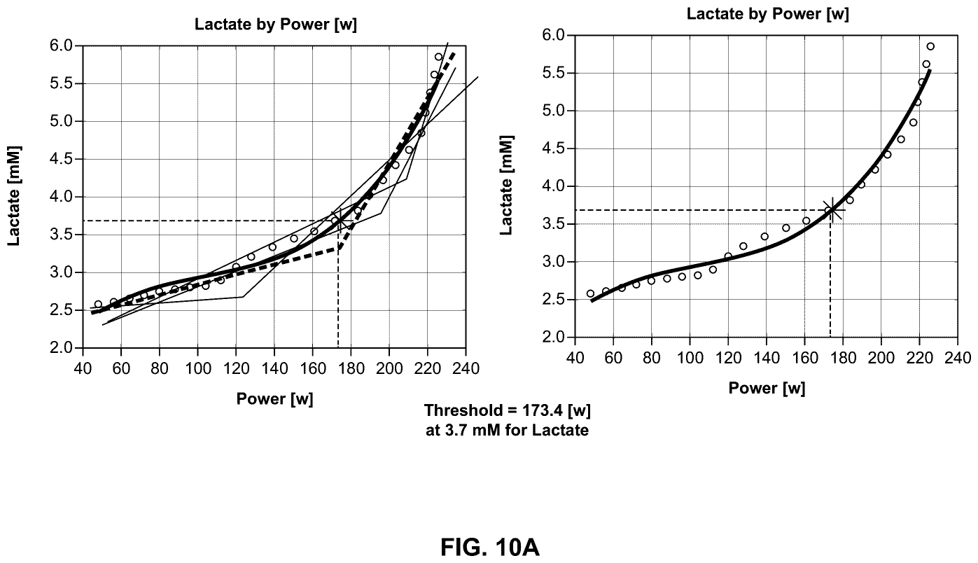

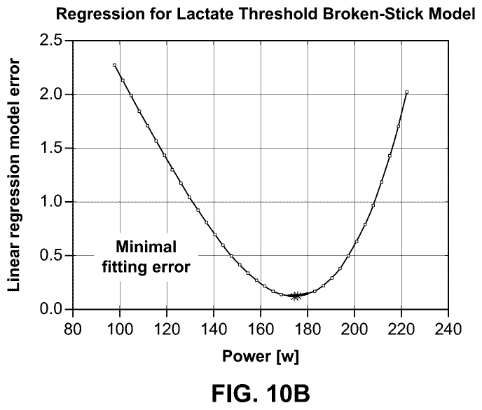

In some embodiments, the processor is configured to determine an anaerobic threshold based on the plurality of lactate concentrations using a piecewise linear regression model. In some embodiments, the processor is configured to determine an anaerobic threshold based on the plurality of lactate concentrations using a broken stick model as shown in FIGS. 10A-10B. In some embodiments, the broken stick model can comprise dividing the plurality of lactate concentrations into two groups of data and conducting a regression analysis to find a best fit line for each group of data connected at a breakpoint. In some embodiments, finding a best fit line for each group of data can comprise minimizing the sum of the squared error (SSE), also known as the residual sum of squares (RSS), for example across both groups of data points, by varying the membership of data points in each group. In some embodiments, the breakpoint provides the anaerobic threshold in terms of (i) lactate concentration and (ii) power or heart rate.

In some embodiments, the method can comprise repeating the lactate threshold test with smaller incremental increases in terms of power or heart rate to refine the aerobic threshold determination or the anaerobic threshold determination. In some embodiments, the initial lactate threshold test can be conducted by measuring lactate in 25 watt increments and the lactate threshold test can be repeated using a smaller incremental increase, e.g., 20 watt increments or 10 watt increments. In some embodiments, the initial lactate threshold test can be conducted by measuring lactate in 20 watt increments and the lactate threshold test can be repeated using a smaller incremental increase, e.g., 10 watt increments or 5 watt increments.

In some embodiments, the method can further comprise repeating the lactate threshold test in about a week, about two weeks, about three weeks, about 4 weeks, about 5 weeks, about 6 weeks, about 7 weeks, about 8 weeks, about 9 weeks, about 10 weeks, about 12 weeks, about 14 weeks, about 16 weeks, about a month, about two months, about 3 months, about 4 months, about 5 months, about 6 months, about 9 months, or about a year. In some embodiments, the processor is configured to compare the aerobic and anaerobic thresholds determined from the previous lactate threshold test(s) to the results from the recently performed test. The output of the processor can be numerical and/or graphical to track progress. The notification to the wearer of the lactate-responsive sensor or other interested party can be auditory, tactile (haptic), or any combination thereof.

The present disclosure is further illustrated by the following embodiments.

(1) An analyte sensor comprising:
a substrate;
a first working electrode located on the substrate;
a second working electrode located on the substrate;
a lactate-responsive sensing area disposed on a surface of the first working electrode; and
a glucose-responsive sensing area disposed on a surface of the second working electrode;
a first membrane that is permeable to lactate overcoating the lactate-responsive sensing area; and
a second membrane that is permeable to glucose overcoating the glucose-responsive sensing area and the lactate-responsive sensing area,
wherein the sensor is configured to be partially inserted into an individual's skin.

(2) The analyte sensor of (1), wherein the lactate-responsive sensing area comprises lactate oxidase.

(3) The analyte sensor of (1) or (2), wherein the lactate-responsive sensing area comprises a first polymer and a first electron transfer agent.

(4) The analyte sensor of (3), wherein the first electron transfer agent is covalently bonded to the first polymer.

(5) The analyte sensor of (3) or (4), wherein the first electron transfer agent comprises an osmium complex.

(6) The analyte sensor of any one of (3-5), wherein the first polymer is a poly(4-vinylpyridine)-based polymer.

(7) The analyte sensor of any one of (3-6), wherein the lactate-responsive sensing area further comprises a first crosslinker.

(8) The analyte sensor of (7), wherein the first crosslinker is polyethylene glycol diglycidyl ether (PEGDGE).

(9) The analyte sensor of any one of (1-8), wherein the glucose-responsive sensing area comprises glucose oxidase.

(10) The analyte sensor of any one of (1-9), wherein the glucose-responsive sensing area comprises a second polymer and a second electron transfer agent.

(11) The analyte sensor of (10), wherein the second electron transfer agent is covalently bonded to the second polymer.

(12) The analyte sensor of (10) or (11), wherein the second electron transfer agent comprises an osmium complex.

(13) The analyte sensor of any one of (10-12), wherein the second polymer is a poly(4-vinylpyridine)-based polymer.

(14) The analyte sensor of any one of (10-13), wherein the glucose-responsive sensing area further comprises a second crosslinker.

(15) The analyte sensor of (14), wherein the second crosslinker is PEGDGE.

(16) The analyte sensor of (1-15), wherein the sensor further comprises a reference electrode.

(17) The analyte sensor of (1-16), wherein the sensor further comprises a counter electrode.

(18) The analyte sensor of (1-17), wherein the first membrane and the second membrane have different compositions.

(19) The analyte sensor of (1-18), wherein the first membrane comprises a polyvinylpyridine homopolymer or copolymer.

(20) The analyte sensor of (19), wherein the first membrane further comprises a first crosslinking agent.

(21) The analyte sensor of (20), wherein the first crosslinking agent is polyethylene glycol diglycidyl ether (PEGDGE).

(22) The analyte sensor of (1-21), wherein the second membrane comprises a polyvinylpyridine-co-styrene polymer.

(23) The analyte sensor of (22), wherein the second membrane further comprises a second crosslinking agent.

(24) The analyte sensor of (23), wherein the second crosslinking agent is glycerol triglycidyl ether.

(25) A method for monitoring lactate levels in an individual, comprising:
exposing an analyte sensor of a sensing system to a fluid; wherein the analyte sensor comprises:
   a substrate;
   a first working electrode located on the substrate;
   a second working electrode located on the substrate;
   a lactate-responsive sensing area disposed on a surface of the first working electrode; and
   a glucose-responsive sensing area disposed on a surface of the second working electrode;
   a first membrane that is permeable to lactate overcoating the lactate-responsive sensing area; and
   a second membrane that is permeable to glucose overcoating the glucose-responsive sensing area and the lactate-responsive sensing area,
   wherein the sensor is configured to be partially inserted into the individual's skin;
applying a potential to the first working electrode of the analyte sensor;
obtaining a first signal at or above an oxidation-reduction potential of the lactate-responsive sensing area, the signal being proportional to a concentration of lactate in the fluid; and
correlating the signal to the concentration of lactate in the fluid.

(26) The method of (25), further comprising:
obtaining a second signal at or above an oxidation-reduction potential of the glucose-responsive sensing area, the signal being proportional to a concentration of glucose in the fluid; and
correlating the second signal to the concentration of glucose in the fluid.

(27) The method of (25) or (26), wherein the lactate-responsive sensing area comprises lactate oxidase.

(28) The method of any one of (25-27), wherein the lactate-responsive sensing area comprises a first polymer and a first electron transfer agent.

(29) The method of (28), wherein the first electron transfer agent is covalently bonded to the first polymer.

(30) The method of (28) or (29), wherein the first electron transfer agent comprises an osmium complex.

(31) The method of any one of (28-30), wherein the first polymer is a poly(4-vinylpyridine)-based polymer.

(32) The method of any one of (28-31), wherein the lactate-responsive sensing area further comprises a first crosslinker.

(33) The method of (32), wherein the first crosslinker is PEGDGE.

(34) The method of any one of (25-33), wherein the glucose-responsive sensing area comprises glucose oxidase.

(35) The method of any one of (25-34), wherein the glucose-responsive sensing area comprises a second polymer and a second electron transfer agent.

(36) The method of (35), wherein the second electron transfer agent is covalently bonded to the second polymer.

(37) The method of (35) or (36), wherein the second electron transfer agent comprises an osmium complex.

(38) The method of any one of (35-37), wherein the second polymer is a poly(4-vinylpyridine)-based polymer.

(39) The method of any one of (35-38), wherein the glucose-responsive sensing area further comprises a second crosslinker.

(40) The method of (39), wherein the second crosslinker is PEGDGE.

(41) The method of (25-40), wherein the sensor further comprises a reference electrode.

(42) The method of (25-41), wherein the sensor further comprises a counter electrode.

(43) The method of (25-42), wherein the first membrane and the second membrane have different compositions.

(44) The method of (25-43), wherein the first membrane comprises a polyvinylpyridine homopolymer or copolymer.

(45) The method of (44), wherein the first membrane further comprises a first crosslinking agent.

(46) The method of (45), wherein the first crosslinking agent is polyethylene glycol diglycidyl ether (PEGDGE).

(47) The method of (25-46), wherein the second membrane comprises a polyvinylpyridine-co-styrene polymer.

(48) The method of (47), wherein the second membrane further comprises a second crosslinking agent.

(49) The method of (48), wherein the second crosslinking agent is glycerol triglycidyl ether.

(50) A method of determining an anaerobic threshold in an individual, comprising:
continuously measuring signals indicative of lactate concentrations in a biological fluid in an individual with a sensing system comprising a lactate-responsive sensor;
communicating the signals indicative of lactate concentrations measured by the lactate-responsive sensor to a processor;
determining an anaerobic threshold based on the signals indicative of lactate concentrations.

(51) The method of (50), wherein the individual is performing a lactate threshold test.

(52) The method of (51), wherein the lactate threshold test is a step test with incremental increases in power.

(53) The method of any one of (50-52), wherein the anaerobic threshold is determined by the processor using a broken-stick model.

(54) The method of any one of (50-53), wherein the anaerobic threshold is determined by the processor using a D-max method.

(55) The method of any one of (50-54), wherein the anaerobic threshold is determined by the processor using a modified D-max method.

(56) The method of any one of (50-55), wherein continuously measuring signals indicative of lactate concentrations comprises measuring signals indicative of lactate concentrations about every 10 seconds, about every 15 seconds, about every 20 seconds, about every 30 seconds, about every 45 seconds, about every minute, about every 1.5 minutes, about every 2 minutes, about every 3 minutes, about every 5 minutes, about every 10 minutes, about every 15 minutes, about every 20 minutes, about every 30 minutes, about every 45 minutes, about every hour, about every 2 hours, or about every 3 hours.

(57) The method of any one of (50-56), wherein continuously measuring signals indicative of lactate concentrations comprises measuring signals indicative of lactate concentrations about every minute.

(58) The method of any one of (50-55), wherein the lactate-responsive sensor is the sensor of any one of (1-24).

(59) A method of determining an aerobic threshold in an individual, comprising:
continuously measuring signals indicative of lactate concentrations in a biological fluid in the individual with a sensing system comprising a lactate-responsive sensor;
communicating the signals indicative of lactate concentrations measured by the lactate-responsive sensor to a processor;
determining an aerobic threshold based on the signals indicative of lactate concentrations.

(60) The method of (59), wherein the individual is performing a lactate threshold test.

(61) The method of (60), wherein the lactate threshold test is a step test with incremental increases in power.

(62) The method of any one of (59-61), wherein the aerobic threshold is defined as a fixed value.

(63) The method of (62), wherein the aerobic threshold is defined as a baseline lactate concentration plus about 0.5 mM lactate.

(64) The method of (62), wherein the aerobic threshold is defined as a baseline lactate concentration plus about 1 mM lactate.

(65) The method of any one of (59-61), wherein the aerobic threshold is determined by the processor using a log-log model.

(66) The method of any one of (59-61), wherein the aerobic threshold is determined by the processor using a segmented regression analysis.

(67) The method of any one of (59-66), wherein continuously measuring signals indicative of lactate concentrations comprises measuring signals indicative of lactate concentrations about every 10 seconds, about every 15 seconds, about every 20 seconds, about every 30 seconds, about every 45 seconds, about every minute, about every 1.5 minutes, about every 2 minutes, about every 3 minutes, about every 5 minutes, about every 10 minutes, about every 15 minutes, about every 20 minutes, about every 30 minutes, about every 45 minutes, about every hour, about every 2 hours, or about every 3 hours.

(68) The method of any one of (59-67), wherein continuously measuring signals indicative of lactate concentrations comprises measuring signals indicative of lactate concentrations about every minute.

(69) The method of any one of (59-68), wherein the lactate-responsive sensor is the sensor of any one of (1-24).

(70) A sensor control device comprising the sensor of any one of (1-24) and a processor communicatively coupled to the sensor.

(71) The sensor control device of (70), wherein the sensor is configured to continuously measure signals indicative of lactate concentrations in the individual's biological fluid and communicate the signals indicative of lactate concentrations to the processor.

(72) The sensor control device of (71), wherein the processor is configured to determine an aerobic threshold, an anaerobic threshold, or both, based on the signals indicative of lactate concentrations.

(73) The sensor control device of (72), wherein the user is performing a lactate threshold test.

(74) The sensor control device of (73), wherein the lactate threshold test is a step test with incremental increases in power.

(75) The sensor control device of any one of (72-74), wherein the anaerobic threshold is determined by the processor using a broken-stick model.

(76) The sensor control device of any one of (72-74), wherein the anaerobic threshold is determined by the processor using a D-max method.

(77) The sensor control device of any one of (72-74), wherein the anaerobic threshold is determined by the processor using a modified D-max method.

(78) The sensor control device of any one of (72-77), wherein the aerobic threshold is defined as a fixed value.

(79) The sensor control device of (79), wherein the aerobic threshold is defined as a baseline lactate concentration plus about 0.5 mM lactate.

(80) The sensor control device of (79), wherein the aerobic threshold is defined as a baseline lactate concentration plus about 1 mM lactate.

(81) The sensor control device of any one of (72-78), wherein the aerobic threshold is determined by the processor using a log-log model.

(82) The sensor control device of any one of (72-78), wherein the aerobic threshold is determined by the processor using a segmented regression analysis.

(83) The sensor control device of any one of (70-82), wherein the sensor is configured to continuously measure signals indicative of lactate concentrations about every 10 seconds, about every 15 seconds, about every 20 seconds, about every 30 seconds, about every 45 seconds, about every minute, about every 1.5 minutes, about every 2 minutes, about every 3 minutes, about every 5 minutes, about every 10 minutes, about every 15 minutes, about every 20 minutes, about every 30 minutes, about every 45 minutes, about every hour, about every 2 hours, or about every 3 hours.

(84) The sensor control device of any one of (70-83), wherein the sensor is configured to continuously measure signals indicative of lactate concentrations about every minute.

(85) A sensing system comprising the sensor control device of any one of (70-84) and a reader device.

(86) An analyte sensor comprising:
a proximal portion configured to be positioned above a user's skin, and
a distal portion configured to be transcutaneously positioned through the user's skin, the distal portion comprising:
   a substrate;
   a first working electrode located on the substrate;
   a second working electrode located on the substrate;
   a lactate-responsive sensing area disposed on a surface of the first working electrode; and
   a glucose-responsive sensing area disposed on a surface of the second working electrode;
   a first membrane that is permeable to lactate overcoating the lactate-responsive sensing area; and
   a second membrane that is permeable to glucose overcoating the glucose-responsive sensing area and the lactate-responsive sensing area.

(87) The analyte sensor of (86), wherein the lactate-responsive sensing area comprises lactate oxidase.

(88) The analyte sensor of (86) or (87), wherein the lactate-responsive sensing area comprises a first polymer and a first electron transfer agent.

(89) The analyte sensor of (88), wherein the first electron transfer agent is covalently bonded to the first polymer.

(90) The analyte sensor of (88) or (89), wherein the first electron transfer agent comprises an osmium complex.

(91) The analyte sensor of any one of (88-90), wherein the first polymer is a poly(4-vinylpyridine)-based polymer.

(92) The analyte sensor of any one of (88-91), wherein the lactate-responsive sensing area further comprises a first crosslinker.

(93) The analyte sensor of (92), wherein the first crosslinker is PEGDGE.

(94) The analyte sensor of any one of (86-93), wherein the glucose-responsive sensing area comprises glucose oxidase.

(95) The analyte sensor of any one of (86-94), wherein the glucose-responsive sensing area comprises a second polymer and a second electron transfer agent.

(96) The analyte sensor of (95), wherein the second electron transfer agent is covalently bonded to the second polymer.

(97) The analyte sensor of (95) or (96), wherein the second electron transfer agent comprises an osmium complex.

(98) The analyte sensor of any one of (95-97), wherein the second polymer is a poly(4-vinylpyridine)-based polymer.

(99) The analyte sensor of any one of (95-98), wherein the glucose-responsive sensing area further comprises a second crosslinker.

(100) The analyte sensor of (99), wherein the second crosslinker is PEGDGE.

(101) The analyte sensor of (86-100), wherein the sensor further comprises a reference electrode.

(102) The analyte sensor of (86-101), wherein the sensor further comprises a counter electrode.

(103) The analyte sensor of (86-102), wherein the first membrane and the second membrane have different compositions.

(104) The analyte sensor of (86-103), wherein the first membrane comprises a polyvinylpyridine homopolymer or copolymer.

(105) The analyte sensor of (104), wherein the first membrane further comprises a first crosslinking agent.

(106) The analyte sensor of (105), wherein the first crosslinking agent is PEGDGE.

(107) The analyte sensor of (86-106), wherein the second membrane comprises a polyvinylpyridine-co-styrene polymer.

(108) The analyte sensor of (107), wherein the second membrane further comprises a second crosslinking agent.

(109) The analyte sensor of (108), wherein the second crosslinking agent is glycerol triglycidyl ether.

(110) A method for monitoring lactate levels in a user, comprising:
exposing an analyte sensor of a sensing system to a fluid, the analyte sensor comprising a proximal portion configured to be positioned above the user's skin, and a distal portion configured to be transcutaneously positioned through the user's skin, the distal portion comprising:
   a substrate;
   a first working electrode located on the substrate;
   a second working electrode located on the substrate;
   a lactate-responsive sensing area disposed on a surface of the first working electrode; and
   a glucose-responsive sensing area disposed on a surface of the second working electrode;
   a first membrane that is permeable to lactate overcoating the lactate-responsive sensing area; and
   a second membrane that is permeable to glucose overcoating the glucose-responsive sensing area and the lactate-responsive sensing area;
applying a potential to the first working electrode of the analyte sensor;
obtaining a first signal at or above an oxidation-reduction potential of the lactate-responsive sensing area, the signal being proportional to a concentration of lactate in the fluid; and
correlating the signal to the concentration of lactate in the fluid.

(111) The method of (110), further comprising:
obtaining a second signal at or above an oxidation-reduction potential of the glucose-responsive sensing area, the signal being proportional to a concentration of glucose in the fluid; and
correlating the second signal to the concentration of glucose in the fluid.

(112) The method of (110) or (111), wherein the lactate-responsive sensing area comprises lactate oxidase.

(113) The method of any one of (110-112), wherein the lactate-responsive sensing area comprises a first polymer and a first electron transfer agent.

(114) The method of (113), wherein the first electron transfer agent is covalently bonded to the first polymer.

(115) The method of (113) or (114), wherein the first electron transfer agent comprises an osmium complex.

(116) The method of any one of (113-115), wherein the first polymer is a poly(4-vinylpyridine)-based polymer.

(117) The method of any one of (113-116), wherein the lactate-responsive sensing area further comprises a first crosslinker.

(118) The method of (115), wherein the first crosslinker is PEGDGE.

(119) The method of any one of (110-118), wherein the glucose-responsive sensing area comprises glucose oxidase.

(120) The method of any one of (110-119), wherein the glucose-responsive sensing area comprises a second polymer and a second electron transfer agent.

(121) The method of (120), wherein the second electron transfer agent is covalently bonded to the second polymer.

(122) The method of (120) or (121), wherein the second electron transfer agent comprises an osmium complex.

(123) The method of any one of (120-122), wherein the second polymer is a poly(4-vinylpyridine)-based polymer.

(124) The method of any one of (120-123), wherein the glucose-responsive sensing area further comprises a second crosslinker.

(125) The method of (124), wherein the second crosslinker is PEGDGE.

(126) The method of (110-125), wherein the sensor further comprises a reference electrode.

(127) The method of (110-126), wherein the sensor further comprises a counter electrode.

(128) The method of (110-127), wherein the first membrane and the second membrane have different compositions.

(129) The method of (110-128), wherein the first membrane comprises a polyvinylpyridine homopolymer or copolymer.

(130) The method of (129), wherein the first membrane further comprises a first crosslinking agent.

(131) The method of (130), wherein the first crosslinking agent is polyethylene glycol diglycidyl ether (PEGDGE).

(132) The method of (110-131), wherein the second membrane comprises a polyvinylpyridine-co-styrene polymer.

(133) The method of (132), wherein the second membrane further comprises a second crosslinking agent.

(134) The method of (133), wherein the second crosslinking agent is glycerol triglycidyl ether.

(135) A method of determining an anaerobic threshold in an individual, comprising:
continuously measuring signals indicative of lactate concentrations in a biological fluid in an individual with a sensing system comprising a lactate-responsive sensor;
communicating the signals indicative of lactate concentrations measured by the lactate-responsive sensor to a processor; and
determining an anaerobic threshold based on the signals indicative of lactate concentrations by the processor.

(136) The method of (135), wherein the individual is performing a lactate threshold test.

(137) The method of (136), wherein the lactate threshold test is a step test with incremental increases in power.

(138) The method of any one of (135-137), wherein the anaerobic threshold is determined by the processor using a broken-stick model.

(139) The method of any one of (135-137), wherein the anaerobic threshold is determined by the processor using a D-max method.

(140) The method of any one of (135-137), wherein the anaerobic threshold is determined by the processor using a modified D-max method.

(141) The method of any one of (135-140), wherein continuously measuring signals indicative of lactate concentrations comprises measuring signals indicative of lactate concentrations about every 10 seconds, about every 15 seconds, about every 20 seconds, about every 30 seconds, about every 45 seconds, about every minute, about every 1.5 minutes, about every 2 minutes, about every 3 minutes, about every 5 minutes, about every 10 minutes, about every 15 minutes, about every 20 minutes, about every 30 minutes, about every 45 minutes, about every hour, about every 2 hours, or about every 3 hours.

(142) The method of any one of (135-141), wherein continuously measuring signals indicative of lactate concentrations comprises measuring signals indicative of lactate concentrations about every minute.

(143) The method of any one of (135-142), wherein the lactate-responsive sensor is the sensor of any one of (1-24).

(144) The method of any one of (135-142), wherein the lactate-responsive sensor is the sensor of any one of (86-109).

(145) A method of determining an aerobic threshold in an individual, comprising:
continuously measuring signals indicative of lactate concentrations in a biological fluid in the individual with a sensing system comprising a lactate-responsive sensor;
communicating the signals indicative of lactate concentrations to a processor;
determining an aerobic threshold based on the signals indicative of lactate concentrations.

(146) The method of (145), wherein the individual is performing a lactate threshold test.

(147) The method of (146), wherein the lactate threshold test is a step test with incremental increases in power.

(148) The method of any one of (145-147), wherein the aerobic threshold is defined as a fixed value.

(149) The method of (148), wherein the aerobic threshold is defined as a baseline lactate concentration plus about 0.5 mM lactate.

(150) The method of (148), wherein the aerobic threshold is defined as a baseline lactate concentration plus about 1 mM lactate.

(151) The method of any one of (145-147), wherein the aerobic threshold is determined by the processor using a log-log model.

(152) The method of any one of (145-147), wherein the aerobic threshold is determined by the processor using a segmented regression analysis.

(153) The method of any one of (145-152), wherein continuously measuring signals indicative of lactate concentrations comprises measuring signals indicative of lactate concentrations about every 10 seconds, about every 15 seconds, about every 20 seconds, about every 30 seconds, about every 45 seconds, about every minute, about every 1.5 minutes, about every 2 minutes, about every 3 minutes, about every 5 minutes, about every 10 minutes, about every 15 minutes, about every 20 minutes, about every 30 minutes, about every 45 minutes, about every hour, about every 2 hours, or about every 3 hours.

(154) The method of any one of (145-153), wherein continuously measuring signals indicative of lactate concentrations comprises measuring signals indicative of lactate concentrations about every minute.

(155) The method of any one of (145-154), wherein the lactate-responsive sensor is the sensor of any one of (1-24).

(156) The method of any one of (145-154), wherein the lactate-responsive sensor is the sensor of any one of (86-109).

(157) A sensor control device comprising the sensor of any one of (81-104) and a processor communicatively coupled to the sensor.

(158) The sensor control device of (157), wherein the sensor is configured to continuously measure signals indicative of lactate concentrations in the user's biological fluid and communicate the signals indicative of lactate concentrations to the processor.

(159) The sensor control device of (158), wherein the processor is configured to determine an aerobic threshold, an anaerobic threshold, or both, based on the signals indicative of lactate concentrations.

(160) The sensor control device of (159), wherein the user is performing a lactate threshold test.

(161) The sensor control device of (160), wherein the lactate threshold test is a step test with incremental increases in power.

(162) The sensor control device of any one of (159-161), wherein the anaerobic threshold is determined by the processor using a broken-stick model.

(163) The sensor control device of any one of (159-161), wherein the anaerobic threshold is determined by the processor using a D-max method.

(164) The sensor control device of any one of (159-161), wherein the anaerobic threshold is determined by the processor using a modified D-max method.

(165) The sensor control device of any one of (159-164), wherein the aerobic threshold is defined as a fixed value.

(166) The sensor control device of (165), wherein the aerobic threshold is defined as a baseline lactate concentration plus about 0.5 mM lactate.

(167) The sensor control device of (153), wherein the aerobic threshold is defined as a baseline lactate concentration plus about 1 mM lactate.

(168) The sensor control device of any one of (159-164), wherein the aerobic threshold is determined by the processor using a log-log model.

(169) The sensor control device of any one of (159-164), wherein the aerobic threshold is determined by the processor using a segmented regression analysis.

(170) The sensor control device of any one of (157-169), wherein the sensor is configured to continuously measure signals indicative of lactate concentrations about every 10 seconds, about every 15 seconds, about every 20 seconds, about every 30 seconds, about every 45 seconds, about every minute, about every 1.5 minutes, about every 2 minutes, about every 3 minutes, about every 5 minutes, about every 10 minutes, about every 15 minutes, about every 20 minutes, about every 30 minutes, about every 45 minutes, about every hour, about every 2 hours, or about every 3 hours.

(171) The sensor control device of any one of (157-170), wherein the sensor is configured to continuously measure signals indicative of lactate concentrations about every minute.

(172) A sensing system comprising the sensor control device of any one of (157-171).

(173) A sensor control device comprising:
a lactate-responsive sensor comprising a proximal portion configured to be positioned above a user's skin and a distal portion configured to be transcutaneously positioned through the user's skin and in contact with the user's biological fluid, wherein the lactate-responsive sensor is configured to continuously measure signals indicative of lactate concentrations in the user's biological fluid and communicate the signals indicative of lactate concentrations to a processor;
the processor configured to determine an aerobic threshold, an anaerobic threshold, or both, based on the signals indicative of lactate concentrations.

(174) The sensor control device of (173), wherein the user is performing a lactate threshold test.

(175) The sensor control device of (174), wherein the lactate threshold test is a step test with incremental increases in power.

(176) The sensor control device of any one of (173-175), wherein the anaerobic threshold is determined by the processor using a broken-stick model.

(177) The sensor control device of any one of (173-175), wherein the anaerobic threshold is determined by the processor using a D-max method.

(178) The sensor control device of any one of (173-175), wherein the anaerobic threshold is determined by the processor using a modified D-max method.

(179) The sensor control device of any one of (173-168), wherein the aerobic threshold is defined as a fixed value.

(180) The sensor control device of (179), wherein the aerobic threshold is defined as a baseline lactate concentration plus about 0.5 mM lactate.

(181) The sensor control device of (179), wherein the aerobic threshold is defined as a baseline lactate concentration plus about 1 mM lactate.

(182) The sensor control device of any one of (173-178), wherein the aerobic threshold is determined by the processor using a log-log model.

(183) The sensor control device of any one of (173-178), wherein the aerobic threshold is determined by the processor using a segmented regression analysis.

(184) The sensor control device of any one of (173-183), wherein the sensor is configured to continuously measure signals indicative of lactate concentrations about every 10 seconds, about every 15 seconds, about every 20 seconds, about every 30 seconds, about every 45 seconds, about every minute, about every 1.5 minutes, about every 2 minutes, about every 3 minutes, about every 5 minutes, about every 10 minutes, about every 15 minutes, about every 20 minutes, about every 30 minutes, about every 45 minutes, about every hour, about every 2 hours, or about every 3 hours.

(185) The sensor control device of any one of (173-184), wherein the sensor is configured to continuously measure signals indicative of lactate concentrations about every minute.

(186) The sensor control device of any one of (173-185), wherein the distal portion comprising:
a substrate;
a first working electrode located on the substrate;
a second working electrode located on the substrate;
a lactate-responsive sensing area disposed on a surface of the first working electrode; and
a glucose-responsive sensing area disposed on a surface of the second working electrode;
a first membrane that is permeable to lactate overcoating the lactate-responsive sensing area; and
a second membrane that is permeable to glucose overcoating the glucose-responsive sensing area and the lactate-responsive sensing area.

(187) The sensor control device of (186), wherein the lactate-responsive sensing area comprises lactate oxidase.

(188) The sensor control device of (186) or (187), wherein the lactate-responsive sensing area comprises a first polymer and a first electron transfer agent.

(189) The sensor control device of (188), wherein the first electron transfer agent is covalently bonded to the first polymer.

(190) The sensor control device of (188) or (189), wherein the first electron transfer agent comprises an osmium complex.

(191) The sensor control device of any one of (188-190), wherein the first polymer is a poly(4-vinylpyridine)-based polymer.

(192) The sensor control device of any one of (188-191), wherein the lactate-responsive sensing area further comprises a first crosslinker.

(193) The sensor control device of (192), wherein the first crosslinker is PEGDGE.

(194) The sensor control device of any one of (186-193), wherein the glucose-responsive sensing area comprises glucose oxidase.

(195) The sensor control device of any one of (186-194), wherein the glucose-responsive sensing area comprises a second polymer and a second electron transfer agent.

(196) The sensor control device of (195), wherein the second electron transfer agent is covalently bonded to the second polymer.

(197) The sensor control device of (195) or (196), wherein the second electron transfer agent comprises an osmium complex.

(198) The sensor control device of any one of (195-197), wherein the second polymer is a poly(4-vinylpyridine)-based polymer.

(199) The sensor control device of any one of (195-198), wherein the glucose-responsive sensing area further comprises a second crosslinker.

(200) The sensor control device of (199), wherein the second crosslinker is PEGDGE.

(201) The sensor control device of (186-200), wherein the sensor further comprises a reference electrode.

(202) The sensor control device of (186-201), wherein the sensor further comprises a counter electrode.

(203) The sensor control device of (186-202), wherein the first membrane and the second membrane have different compositions.

(204) The sensor control device of (186-203), wherein the first membrane comprises a polyvinylpyridine homopolymer or copolymer.

(205) The sensor control device of (204), wherein the first membrane further comprises a first crosslinking agent.

(206) The sensor control device of (205), wherein the first crosslinking agent is PEGDGE.

(207) The sensor control device of (186-206), wherein the second membrane comprises a polyvinylpyridine-co-styrene polymer.

(208) The sensor control device of (207), wherein the second membrane further comprises a second crosslinking agent.

(209) The sensor control device of (208), wherein the second crosslinking agent is glycerol triglycidyl ether.

### EXAMPLES

The example presented below is provided for the purpose of illustration only and the embodiments described herein should in no way be construed as being limited to this example. Rather, the embodiments should be construed to encompass any and all variations which become evident as a result of the teaching provided herein.

### Example 1

A post hoc analysis of the results of 103 individuals and 286 evaluable sensors in 4 previous clinical studies was conducted to determine the accuracy of anaerobic lactate threshold (LT2) determinations with simultaneous glucose monitoring using a subcutaneous lactate/glucose dual biosensor. The lactate/glucose sensor design is shown in FIG. 6. In order to be eligible for participation in the clinical studies, individuals must have, at the time of the study, routinely exercised a minimum of about 150 minutes a week of moderate intensity or a minimum of approximately 75 minutes a week of vigorous intensity aerobic physical activity and agreed to perform moderate and/or vigorous physical activity during the in-clinic visits, as required. Additionally, there was a preference for individuals who regularly used bikes in their exercise regimen.

The individuals performed one of two tests on a stationary bike as discussed below. The first test was based on an incremental exercise protocol that included the following phases:
Baseline Phase: established the level of lactate at rest before individuals started the required exercise routine.
Incremental effort phase: subjects completed up to 8 intervals of increasing intensity (from 30 watts to 240 watts in 30-watt steps). Individuals proceeded to the sustained effort phase after (i) lactate level was 5.5 mM or higher, or (ii) they completed all 8 intervals. Individuals stopped at any time upon their request or based on the investigator's discretion.
a Sustained effort phase. Subjects completed 2 intervals at approximately 850 of the maximum pace that they can sustain or 850% of the maximum effort attained when lactate level of 5.5 mM was determined by the sensor.
Cool-down phase: lactate monitoring was continued while lactate levels declined during cool-down exercise.
Rest phase: lactate monitoring was continued until lactate levels returned to baseline.

The incremental exercise protocol is summarized in Table 1.

TABLE 1
Incremental Exercise Protocol

| | | Time | |
|---|---|---|---|
| Phases | Interval | (Minutes) | Activity |
| Baseline | 1 | 0 | Baseline Blood sample 1 |
| | 2 | 5:00 | Baseline Blood sample 2 |
| | 3 | 5:00 | Baseline Blood sample 3 |
| Incremental | 4 | 4:00 | 30 watts warm-up |
| Effort | | | |
| | | 1:00 | YSI Blood sample 2 (easy pedal) |
| | 5 | 4:00 | 60 watts |
| | | 1:00 | YSI Blood sample 3 (easy pedal) |
| | 6 | 4:00 | 90 watts |
| | | 1:00 | YSI Blood sample 4 (easy pedal) |
| | 7 | 4:00 | 120 watts |
| | | 1:00 | YSI Blood sample 5 (easy pedal) |
| | 8 | 4:00 | 150 watts |
| | | 1:00 | YSI Blood sample 6 (easy pedal) |
| | 9 | 4:00 | 180 watts |
| | | 1:00 | YSI Blood sample 7 (easy pedal) |
| | 10 | 4:00 | 210 watts |
| | | 1:00 | YSI Blood sample 8 (easy pedal) |
| | 11 | 4:00 | 240 watts |
| | | 1:00 | YSI Blood sample 9 (easy pedal) |
| Sustained | 12 | 4:00 | 85% of max power reached (lookup |
| Effort | | | table) |
| | | 1:00 | YSI Blood sample 10 (easy pedal) |
| | 13 | 4:00 | 85% of max power reached (lookup |
| | | | table) |
| | | 1:00 | YSI Blood sample 11 (easy pedal) |
| Cool-down | 14 | 4:00 | 50 watts |
| | | 1:00 | YSI Blood sample 12 (easy pedal) |
| | 15 | 4:00 | 50 watts |
| | | 1:00 | YSI Blood sample 13 (begin rest) |
| Post-exercise | 16-20 | | Continue blood sampling every |
| rest | | | 10 minutes until baseline lactate |
| | | | reached or 50 minutes. |

The second test was based on an endurance exercise protocol that included the following phases:
Baseline Phase: established the level of lactate at rest before individuals started the required exercise routine.
Exercise phase: subjects started the exercise at 30 W, and then progressed to the next power setting in 5 minute intervals (60, 90, 120, 150, 180, 210, and 240 W) unless the lactate level reached >2.6 mM. If the lactate level was ≥2.6 mM but <3.1 mM, power setting for the next step was +10W from the step just completed. If the lactate level was ≥3.1 mM but ≤4.5 mM, power setting for the next step was dependent on the lactate prior to this lactate result. Specifically, if the prior lactate result was <3.1 mM (i.e., lactate level rose from prior lactate result), power setting for the next step was −10 W from the step just completed. If the prior lactate result was between about 3.1˜4.5 mM (i.e., lactate level was steady relative to the prior lactate result) or >4.5 mM (i.e., lactate level dropped from the prior lactate result), power setting for the next step was the same as the step just completed.
Rest phase: lactate monitoring was continued until lactate levels returned to baseline.

The endurance exercise protocol is summarized in Table 2.

TABLE 2
Endurance Exercise Protocol

| | | Time | |
|---|---|---|---|
| Phases | Interval | (Minutes) | Activity |
| Baseline | 1 | 0 | Baseline Blood sample 1 |
| | 2 | 5:00 | Baseline Blood sample 2 |
| | 3 | 5:00 | Baseline Blood sample 3 |
| Exercise | 4 | 4:00 | 50 W |
| Effort | | | |
| | | 1:00 | YSI Blood sample 4 (easy pedal) |
| | 5 | 4:00 | 90 W or power adjust from last step |
| | | 1:00 | YSI Blood sample 5 (easy pedal) |
| | 6 | 4:00 | 120 W or power adjust from last step |
| | | 1:00 | YSI Blood sample 6 (easy pedal) |
| | 7 | 4:00 | 150 W or power adjust from last step |
| | | 1:00 | YSI Blood sample 7 (easy pedal) |
| | 8 | 4:00 | 180 W or power adjust from last step |
| | | 1:00 | YSI Blood sample 8 (easy pedal) |
| | 9 | 4:00 | 210 W or power adjust from last step |
| | | 1:00 | YSI Blood sample 9 (easy pedal) |
| | 10 | 4:00 | 240 W or power adjust from last step |
| | | 1:00 | YSI Blood sample 10 (easy pedal) |
| | 11 | 4:00 | 270 W or power adjust from last step |
| | | 1:00 | YSI Blood sample 11 (easy pedal) |
| | 12 | 4:00 | 300 W or power adjust from last step |
| | | 1:00 | YSI Blood sample 12 (easy pedal) |
| Post-exercise | 13-18 | | Continue blood sampling every |
| rest | | | 10 minutes until baseline lactate |
| | | | reached or 1 hour. |

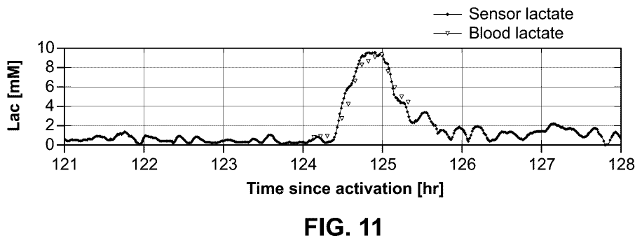

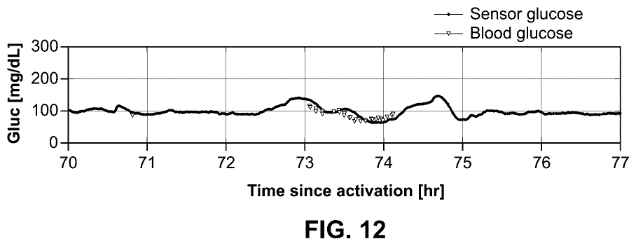

FIG. 11 shows a graph of lactate level for an individual as detected by the sensor over 128 hours compared to a blood lactate test using a venous blood sample. FIG. 12 shows a graph of glucose level for the same individual as detected by the sensor over 77 hours compared to a blood glucose test. The blood glucose test was performed using a YSI analyzer. The sensor was worn for 15 days and detected lactate and glucose levels at a high correlation to those measured with blood samples.

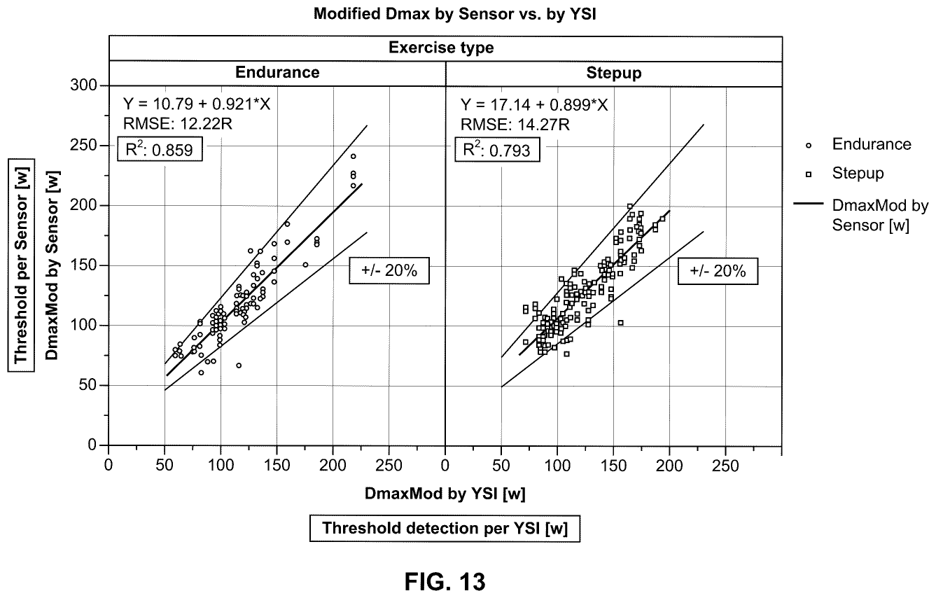

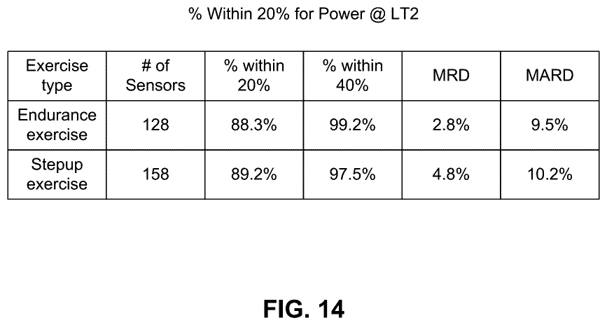

As shown in FIG. 13, anaerobic threshold measurements from the sensor have a high correlation (R² at around 0.8) compared to those measured from the blood samples. Over 88% of the anaerobic threshold measurements from the sensor are within 20% of the anaerobic threshold measurements from the blood lactate test with a low absolute error of about 10% (FIG. 14).

### Example 2

### Formulations for Lactate-Responsive Sensing Area Deposition:

Lactate oxidase was combined with an osmium-containing poly(4-vinylpyridine)-based polymer (Os-PVP) in aqueous solution formulations as specified in Tables 3 and 4 shown below.

TABLE 3
Formulation 1

| | in 10 mM MES | |
|---|---|---|
| | (pH 5.5) | Volume |
| Component | mg/mL | mL |
| Buffer | | |
| LOX | 80 | 0.32 |
| HSA | 80 | 0.32 |
| Os-PVP | 40 | 0.24 |
| PEGDGE400 | 40 | 0.16 |

TABLE 4
Formulation 2

| | in 10 mM MES | | Final |
|---|---|---|---|
| | (pH 5.5) | Volume | Concentration |
| Component | mg/mL | mL | mg/mL |
| Buffer | | | 10 mM |
| LOX | 130 | 0.32 | 40 |
| HSA | 60 | 0.32 | 18.5 |
| Os-PVP | 40 | 0.24 | 9.2 |
| PEGDGE400 | 40 | 0.16 | 6.2 |

It is to be appreciated that the Detailed Description section, and not the Summary and Abstract sections, is intended to be used to interpret the claims. The Summary and Abstract sections can set forth one or more but not all exemplary embodiments of the present invention as contemplated by the inventor(s), and thus, are not intended to limit the present invention and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

The claims in the instant application are different than those of the parent application or other related applications. The Applicant therefore rescinds any disclaimer of claim scope made in the parent application or any predecessor application in relation to the instant application. The Examiner is therefore advised that any such previous disclaimer and the cited references that it was made to avoid, can need to be revisited. Further, the Examiner is also reminded that any disclaimer made in the instant application should not be read into or against the parent application.

As used herein, the phrase “in some embodiments” in relation to a feature means that the feature may be present in any embodiment, unless the feature is obviously technically incompatible with that embodiment. Furthermore, any subset of features of one embodiment can be combined with any subset of features from any other embodiment(s) in any combination, unless such combination is obviously technically incompatible.

1. An analyte sensor for detecting glucose and lactate in vivo, the sensor comprising:
a proximal portion configured to be positioned above a user's skin; and a distal portion configured to be transcutaneously positioned through the user's skin such that the distal portion is in contact with the user's interstitial fluid to detect glucose and lactate in vivo; the distal portion comprising:
a substrate;
a first working electrode located on the substrate;
a second working electrode located on the substrate;
a lactate-responsive sensing area disposed on a surface of the first working electrode; and
a glucose-responsive sensing area disposed on a surface of the second working electrode;
a first membrane that is permeable to lactate overcoating the lactate-responsive sensing area; and
a second membrane that is permeable to glucose overcoating the glucose-responsive sensing area and the lactate-responsive sensing area.

2. The analyte sensor of claim 1, wherein the lactate-responsive sensing area comprises lactate oxidase.

3. The analyte sensor of claim 1, wherein the lactate-responsive sensing area comprises a first polymer and a first electron transfer agent.

4. The analyte sensor of claim 3, wherein the first electron transfer agent is covalently bonded to the first polymer.

5. The analyte sensor of claim 1, wherein the glucose-responsive sensing area comprises glucose oxidase.

6. The analyte sensor of claim 1, wherein the glucose-responsive sensing area comprises a second polymer and a second electron transfer agent.

7. The analyte sensor of claim 6, wherein the second electron transfer agent is covalently bonded to the second polymer.

8. The analyte sensor of claim 1, wherein the sensor tail-further comprises a reference electrode and a counter electrode.

9. The analyte sensor of claim 1, wherein the first membrane and the second membrane have different compositions.

10. A method for monitoring lactate levels in an individual, comprising:
exposing the analyte sensor of claim 1 to interstitial fluid;
applying a potential to the first working electrode of the analyte sensor;
obtaining a first signal at or above an oxidation-reduction potential of the lactate-responsive sensing area, the signal being proportional to a concentration of lactate in the interstitial fluid; and
correlating the signal to the concentration of lactate in the interstitial fluid.

11. The method of claim 10, further comprising:
obtaining a second signal at or above an oxidation-reduction potential of the glucose-responsive sensing area, the signal being proportional to a concentration of glucose in the fluid; and
correlating the second signal to the concentration of glucose in the interstitial fluid.

12. (canceled)

13. (canceled)

14. (canceled)

15. (canceled)

16. (canceled)

17. (canceled)

18. (canceled)

19. (canceled)

20. (canceled)

21. A method of determining an anaerobic threshold in an individual, comprising:
continuously measuring signals indicative of lactate concentrations in a biological fluid in the individual with a sensing system comprising the analyte sensor of claim 1;
communicating the signals indicative of lactate concentrations measured by the analyte sensor to a processor;
determining an anaerobic threshold based on the signals indicative of lactate concentrations.

22. The method of claim 21, wherein the individual is performing a lactate threshold test.

23. The method of claim 22, wherein the lactate threshold test is a step test with incremental increases in power.

24. The method of claim 21, wherein the anaerobic threshold is determined by the processor using a broken-stick model.

25. The method of claim 21, wherein the anaerobic threshold is determined by the processor using a D-max method.

26. The method of claim 21, wherein the anaerobic threshold is determined by the processor using a modified D-max method.

27. (canceled)

28. A method of determining an aerobic threshold in an individual, comprising:
continuously measuring signals indicative of lactate concentrations in a biological fluid in the individual with a sensing system comprising the analyte of claim 1;
communicating the signals indicative of lactate concentrations measured by the analyte sensor to a processor;
determining an aerobic threshold based on the signals indicative of lactate concentrations.

29. The method of claim 28, wherein the individual is performing a lactate threshold test.

30. The method of claim 29, wherein the lactate threshold test is a step test with incremental increases in power.

31. The method of claim 28, wherein the aerobic threshold is defined as a fixed value.

32. The method of claim 31, wherein the aerobic threshold is defined as a baseline lactate concentration plus about 0.5 mM lactate.

33. The method of claim 31, wherein the aerobic threshold is defined as a baseline lactate concentration plus about 1 mM lactate.

34. The method of claim 28, wherein the aerobic threshold is determined by the processor using a log-log model.

35. The method of claim 28, wherein the aerobic threshold is determined by the processor using a segmented regression analysis.

36. (canceled)
